(19) United States
(12) Patent Application Publication
(10) Pub. No.: US 2007/0259375 A1
(43) Pub. Date: Nov. 8, 2007

(54) Biomarkers and Methods for Determining Sensitivity to Epidermal Growth Factor Receptor Modulators in Non-Small Cell Lung Cancer

(75) Inventors: Shirin K. Ford, Princeton, NJ (US); Nancy-Anne Perkins, Lawrenceville, NJ (US); Donald G. Jackson, Lawrenceville, NJ (US); Shirin K. Ford, Princeton, NJ (US); Nancy-Anne Perkins, Lawrenceville, NJ (US); Donald G. Jackson, Lawrenceville, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 10/594,211

(22) PCT Filed: Mar. 28, 2005
(86) PCT No.: PCT/US05/10454

§ 371 Date: Jun. 21, 2007

Related U.S. Application Data
(60) Provisional application No. 60/556,903, filed on Mar. 26, 2004.

(51) Int. Cl. G01N 33/53

(52) U.S. Cl. 435/7.1

(57) ABSTRACT

EGFR biomarkers useful in a method for identifying a mammal that will respond therapeutically to a method of treating cancer comprising administering an EGFR modulator, wherein the method comprises (a) exposing a biological sample from the mammal to the EGFR modulator and (b) measuring in the biological sample the level of the at least one biomarker, wherein a difference in the level of the at least one biomarker measured in (b) compared to the level of the biomarker in a mammal that has not been exposed to the EGFR modulator indicates that the mammal will respond therapeutically to the method of treating cancer.

### SEQUENCE LISTING:

A compact disc labeled “Copy 1” contains the Sequence Listing as 10219 PCT.ST25.txt. The Sequence Listing is 1452 KB in size and was recorded Mar. 24, 2005. The compact disk is 1 of 2 compact disks. A duplicate copy of the compact disc is labeled “Copy 2” and is 2 of 2 compact discs.

The compact disc and duplicate copy are identical and are hereby incorporated by reference into the present application.

### FIELD OF THE INVENTION

The present invention relates generally to the field of pharmacogenomics, and more specifically to methods and procedures to determine drug sensitivity in patients to allow the identification of individualized genetic profiles which will aid in treating diseases and disorders.

### BACKGROUND OF THE INVENTION

Cancer is a disease with extensive histoclinical heterogeneity. Although conventional histological and clinical features have been correlated to prognosis, the same apparent prognostic type of tumors varies widely in its responsiveness to therapy and consequent survival of the patient.

New prognostic and predictive markers, which would facilitate an individualization of therapy for each patient, are needed to accurately predict patient response to treatments, such as small molecule or biological molecule drugs, in the clinic. The problem may be solved by the identification of new parameters that could better predict the patient's sensitivity to treatment. The classification of patient samples is a crucial aspect of cancer diagnosis and treatment. The association of a patient's response to a treatment with molecular and genetic markers can open up new opportunities for treatment development in non-responding patients, or distinguish a treatment's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (M. Cockett et al., 2000, Current Opinion in Biotechnology, 11:602-609).

The ability to predict drug sensitivity in patients is particularly challenging because drug responses reflect not only properties intrinsic to the target cells, but also a host's metabolic properties. Efforts to use genetic information to predict drug sensitivity have primarily focused on individual genes that have broad effects, such as the multidrug resistance genes, mdr1 and mrp1 (P. Solmeveld, 2000, J. Intern. Med., 247:521-534).

The development of microarray technologies for large scale characterization of gene mRNA expression pattern has made it possible to systematically search for molecular markers and to categorize cancers into distinct subgroups not evident by traditional histopathological methods (J. Khan et al., 1998, Cancer Res., 58:5009-5013; A. A. Alizadeh et al., 2000, Nature, 403:503-511; M. Bittner et al., 2000, Nature, 406:536-540; J. Khan et al., 2001, Nature Medicine, 7(6):673-679; and T. R. Golub et al., 1999, Science, 286:531-537; U. Alon et al., 1999, Proc. Natl. Acad. Sci. USA, 96:6745-6750). Such technologies and molecular tools have made it possible to monitor the expression level of a large number of transcripts within a cell population at any given time (see, e.g., Schena et al., 1995, Science, 270:467-470; Lockhart et al., 1996, Nature Biotechnology, 14:1675-1680; Blanchard et al., 1996, Nature Biotechnology, 14:1649; U.S. Pat. No. 5,569,588 to Ashby et al.).

Recent studies demonstrate that gene expression information generated by microarray analysis of human tumors can predict clinical outcome (L. J. van't Veer et al., 2002, Nature, 415:530-536; T. Sorlie et al., 2001, Proc. Natl. Acad. Sci. USA, 98:10869-10874; M. Shipp et al., 2002, Nature Medicine, 8(1):68-74: G. Glinsky et al., 2004, The Journal of Clin. Invest., 113(6):913-923). These findings bring hope that cancer treatment will be vastly improved by better predicting the response of individual tumors to therapy.

Needed are new and alternative methods and procedures to determine drug sensitivity in patients to allow the development of individualized genetic profiles which are necessary to treat diseases and disorders based on patient response at a molecular level.

### SUMMARY OF THE INVENTION

The invention provides methods and procedures for determining patient sensitivity to one or more Epidermal Growth Factor Receptor (EGFR) modulators. The invention also provides methods of determining or predicting whether an individual requiring therapy for a disease state such as cancer will or will not respond to treatment, prior to administration of the treatment, wherein the treatment comprises of one or more EGFR modulators. The one or more EGFR modulators are compounds that can be selected from, for example, one or more EGFR-specific ligands, one or more small molecule EGFR inhibitors, or one or more EGFR binding monoclonal antibodies.

In one aspect, the invention provides a method for identifying a mammal that will respond therapeutically to a method of treating cancer comprising administering of an EGFR modulator, wherein the method comprises: (a) measuring in the mammal the level of at least one biomarker selected from the biomarkers of Table 1; (b) exposing a biological sample from the mammal to the EGFR modulator; (c) following the exposing in step (b), measuring in said biological sample the level of the at least one biomarker, wherein a difference in the level of the at least one biomarker measured in step (c) compared to the level of the at least one biomarker measured in step (a) indicates that the mammal will respond therapeutically to the said method of treating cancer.

A difference in the level of the biomarker that is sufficient to indicate whether the mammal will or will not respond therapeutically to the method of treating cancer can be readily determined by one of skill in the art using known techniques. The increase or decrease in the level of the biomarker can be correlated to determine whether the difference is sufficient to identify a mammal that will respond therapeutically. The difference in the level of the biomarker that is sufficient can, in one aspect, be predetermined prior to determining whether the mammal will respond therapeutically to the treatment. In one aspect, the difference in the level of the biomarker is a difference in the mRNA level (measured, for example, by RT-PCT or a microarray), such as at least a two-fold difference, at least a three-fold difference, or at least a four-fold difference in the level of expression. In another aspect, the difference in the level of the biomarker is determined by IHC. In another aspect, the difference in the level of the biomarker refers to a p-value of <0.05 in Anova analysis. In yet another aspect, the difference is determined in an ELISA assay.

As used herein, respond therapeutically refers to the alleviation or abrogation of the cancer. This means that the life expectancy of an individual affected with the cancer will be increased or that one or more of the symptoms of the cancer will be reduced or ameliorated. The term encompasses a reduction in cancerous cell growth or tumor volume. Whether a mammal responds therapeutically can be measured by many methods well known in the art, such as PET imaging.

The mammal can be, for example, a human, rat, mouse, dog, rabbit, pig sheep, cow, horse, cat, primate, or monkey.

The method of the invention can be, for example, an in vitro method wherein the step of measuring in the mammal the level of at least one biomarker comprises taking a biological sample from the mammal and then measuring the level of the at least one biomarker in the biological sample. The biological sample can comprise, for example, at least one of serum, whole fresh blood, peripheral blood mononuclear cells, frozen whole blood, fresh plasma, frozen plasma, urine, saliva, skin, hair follicle, bone marrow, or tumor tissue.

The level of the at least one biomarker can be, for example, the level of protein and/or mRNA transcript of the at least one biomarker.

In another aspect, the invention provides a method for identifying a mammal that will respond therapeutically to a method of treating cancer comprising administering an EGFR modulator, wherein the method comprises: (a) exposing a biological sample from the mammal to the EGFR modulator; (b) following the exposing of step (a), measuring in said biological sample the level of at least one biomarker selected from the biomarkers of Table 1, wherein a difference in the level of the at least one biomarker measured in step (b), compared to the level of the at least one biomarker in a mammal that has not been exposed to said EGFR modulator, indicates that the mammal will respond therapeutically to said method of treating cancer.

In yet another aspect, the invention provides a method for testing or predicting whether a mammal will respond therapeutically to a method of treating cancer comprising administering an EGFR modulator, wherein the method comprises: (a) measuring in the mammal the level of at least one biomarker selected from the biomarkers of Table 1; (b) exposing the mammal to the EGFR modulator; (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

In another aspect, the invention provides a method for determining whether a compound inhibits EGFR activity in a mammal, comprising: (a) exposing the mammal to the compound; and (b) following the exposing of step (a), measuring in the mammal the level of at least one biomarker selected from the biomarkers of Table 1, wherein a difference in the level of said biomarker measured in step (b), compared to the level of the biomarker in a mammal that has not been exposed to said compound, indicates that the compound inhibits EGFR activity in the mammal.

In yet another aspect, the invention provides a method for determining whether a mammal has been exposed to a compound that inhibits EGFR activity, comprising (a) exposing the mammal to the compound; and (b) following the exposing of step (a), measuring in the mammal the level of at least one biomarker selected from the biomarkers of Table 1, wherein a difference in the level of said biomarker measured in step (b), compared to the level of the biomarker in a mammal that has not been exposed to said compound, indicates that the mammal has been exposed to a compound that inhibits EGFR activity.

In another aspect, the invention provides a method for determining whether a mammal is responding to a compound that inhibits EGFR activity, comprising (a) exposing the mammal to the compound; and (b) following the exposing of step (a), measuring in the mammal the level of at least one biomarker selected from the biomarkers of Table 1, wherein a difference in the level of the at least one biomarker measured in step (b), compared to the level of the at least one biomarker in a mammal that has not been exposed to said compound, indicates that the mammal is responding to the compound that inhibits EGFR activity.

As used herein, “responding” encompasses responding by way of a biological and cellular response, as well as a clinical response (such as improved symptoms, a therapeutic effect, or an adverse event), in a mammal.

The invention also provides an isolated biomarker selected from the biomarkers of Table 1. The biomarkers of the invention comprise sequences selected from the nucleotide and amino acid sequences provided in Table 1 and the Sequence Listing, as well as fragments and variants thereof.

The invention also provides a biomarker set comprising two or more biomarkers selected from the biomarkers of Table 1.

The invention also provides kits for determining or predicting whether a patient would be susceptible or resistant to a treatment that comprises one or more EGFR modulators. The patient may have a cancer or tumor such as, for example, a non-small cell lung cancer (NSCLC) or tumor.

In one aspect, the kit comprises a suitable container that comprises one or more specialized microarrays of the invention, one or more EGFR modulators for use in testing cells from patient tissue specimens or patient samples, and instructions for use. The kit may further comprise reagents or materials for monitoring the expression of a biomarker set at the level of mRNA or protein.

In another aspect, the invention provides a kit comprising two or more biomarkers selected from the biomarkers of Table 1.

In yet another aspect, the invention provides a kit comprising at least one of an antibody and a nucleic acid for detecting the presence of at least one of the biomarkers selected from the biomarkers of Table 1. In one aspect, the kit further comprises instructions for determining whether or not a mammal will respond therapeutically to a method of treating cancer comprising administering a compound that inhibits EGFR activity. In another aspect, the instructions comprise the steps of (a) measuring in the mammal the level of at least one biomarker selected from the biomarkers of Table 1, (b) exposing the mammal to the compound, (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

The invention also provides screening assays for determining if a patient will be susceptible or resistant to treatment with one or more EGFR modulators.

The invention also provides a method of monitoring the treatment of a patient having a disease, wherein said disease is treated by a method comprising administering one or more EGFR modulators.

The invention also provides individualized genetic profiles which are necessary to treat diseases and disorders based on patient response at a molecular level.

The invention also provides specialized microarrays, e.g., oligonucleotide microarrays or cDNA microarrays, comprising one or more biomarkers having expression profiles that correlate with either sensitivity or resistance to one or more EGFR modulators.

The invention also provides antibodies, including polyclonal or monoclonal, directed against one or more biomarkers of the invention.

The invention will be better understood upon a reading of the detailed description of the invention when considered in connection with the accompanying figures.

### BRIEF DESCRIPTION OF THE FIGURES

FIG. 1 illustrates the scheme used for identifying the Table 1 biomarkers.

FIG. 2 illustrates the scheme used for identifying the Table 2 biomarkers.

FIG. 3 shows the mRNA levels of EGFR determined by expression profiling of fourteen NSCLC cell lines.

FIG. 4 illustrates the variance analysis of expression profiles.

FIG. 5 illustrates the variance metric distribution of probe sets for the adenocarcinoma tumors.

FIG. 6 illustrates the variance metric distribution of probe sets for the cell lines.

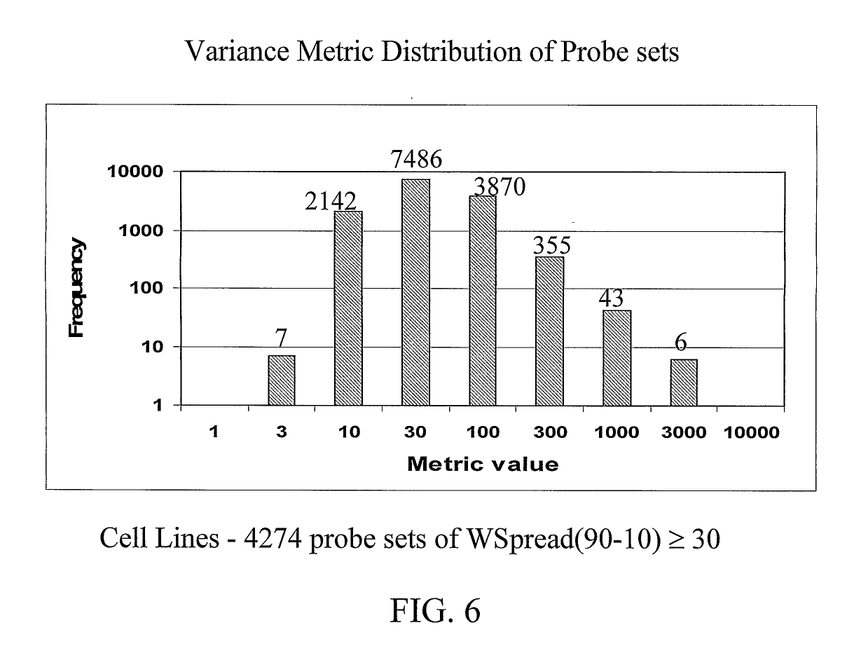

FIG. 7 illustrates the scoring of staining of a Calgranulin B IHC Assay.

### DETAILED DESCRIPTION OF THE INVENTION

Identification of biomarkers that provide rapid and accessible readouts of efficacy, drug exposure, or clinical response is increasingly important in the clinical development of drug candidates. Embodiments of the invention include measuring changes in the levels of secreted proteins, or plasma biomarkers, which represent one category of biomarker. In one aspect, plasma samples, which represent a readily accessible source of material, serves a surrogate tissue for biomarker analysis.

The invention provides biomarkers that respond to the modulation of a specific signal transduction pathway and also correlate with EGFR modulator sensitivity or resistance. These biomarkers can be employed for predicting response to one or more EGFR modulators. In one aspect, the biomarkers of the invention are those provided in Table 1 and the Sequence Listing, including both polynucleotide and polypeptide sequences.

TABLE 1
Biomarkers

| Unigene title and | | Affymetrix |
|---|---|---|
| SEQ ID NO: | Affymetrix Description | Probe Set |
| S100A14: S100 | gb: NM_020672.1 /DEF = Homo sapiens S100- | 218677_at |
| calcium binding | type calcium binding protein A14 (LOC57402), | |
| protein A14 | mRNA. /FEA = mRNA /GEN = LOC57402 | |
| (LOC57402) | /PROD = S100-type calcium binding protein | |
| SEQ ID NOS: 1 | A14 /DB_XREF = gi: 10190711 /UG = Hs.288998 | |
| (DNA) and 148 | S100-type calcium binding protein A14 | |
| (amino acid) | /FL = gb: NM_020672.1 gb: BC005019.1 | |
| | gb: AY007220.1 | |
| JTB: jumping | gb: AF151056.1 /DEF = Homo sapiens HSPC222 | 210434_x_at |
| translocation | mRNA, complete cds. /FEA = mRNA | |
| breakpoint | /PROD = HSPC222 /DB_XREF = gi: 7106833 | |
| (LOC10899) | /UG = Hs.323093 Homo sapiens , jumping | |
| SEQ ID NOS: 2 | translocation breakpoint, clone MGC: 10274, | |
| (DNA) and 149 | mRNA, complete cds /FL = gb: AF151056.1 | |
| (amino acid) | | |
| CDH1: cadherin 1, | gb: NM_004360.1 /DEF = Homo sapiens | 201131_s_at |
| type 1 preproprotein | cadherin 1, type 1, E-cadherin (epithelial) | |
| (LOC999) | (CDH1), mRNA. /FEA = mRNA /GEN = CDH1 | |
| SEQ ID NOS: 3 | /PROD = cadherin 1, type 1, E-cadherin | |
| (DNA) and 150 | (epithelial) /DB_XREF = gi: 4757959 | |
| (amino acid) | /UG = Hs.194657 cadherin 1, type 1, E-cadherin | |
| | (epithelial) /FL = gb: L08599.1 gb: NM_004360.1 | |
| CYR61: cysteine- | gb: NM_001554.1 /DEF = Homo sapiens | 201289_at |
| rich, angiogenic | cysteine-rich, angiogenic inducer, 61 (CYR61), | |
| inducer, 61 | mRNA. /FEA = mRNA /GEN = CYR61 | |
| (LOC3491) | /PROD = cysteine-rich, angiogenic inducer, 61 | |
| SEQ ID NOS: 4 | /DB_XREF = gi: 4504612 /UG = Hs.8867 | |
| (DNA) and 151 | cysteine-rich, angiogenic inducer, 61 | |
| (amino acid) | /FL = gb: BC001271.1 gb: U62015.1 | |
| | gb: AF003594.1 gb: AF031385.1 | |
| | gb: NM_001554.1 | |
| TGFBI: transforming | gb: NM_000358.1 /DEF = Homo sapiens | 201506_at |
| growth factor, beta- | transforming growth factor, beta-induced, 68 kD | |
| induced, 68 kDa | (TGFBI), mRNA. /FEA = mRNA | |
| (LOC7045) | /GEN = TGFBI /PROD = transforming growth | |
| SEQ ID NOS: 5 | factor, beta-induced, 68 kD | |
| (DNA) and 152 | /DB_XREF = gi: 4507466 /UG = Hs.118787 | |
| (amino acid) | transforming growth factor, beta-induced, 68 kD | |
| | /FL = gb: BC000097.1 gb: BC004972.1 | |
| | gb: M77349.1 gb: NM_000358.1 | |
| PSPHL: | Consensus includes gb: BF968134 /FEA = EST | 212509_s_at |
| phosphoserine | /DB_XREF = gi: 12335349 | |
| phosphatase-like | /DB_XREF = est: 602269121F1 | |
| (LOC8781) | /CLONE = IMAGE: 4357349 /UG = Hs.250723 | |
| SEQ ID NOS: 6 | FK506 binding protein 12-rapamycin associated | |
| (DNA) and 153 | protein 1 | |
| (amino acid) | | |
| DKK1: dickkopf | gb: NM_012242.1 /DEF = Homo sapiens | 204602_at |
| homolog 1 | dickkopf (Xenopus laevis) homolog 1 (DKK1), | |
| (LOC22943) | mRNA. /FEA = mRNA /GEN = DKK1 | |
| SEQ ID NOS: 7 | /PROD = dickkopf (Xenopus laevis) homolog 1 | |
| (DNA) and 154 | /DB_XREF = gi: 7110718 /UG = Hs.40499 | |
| (amino acid) | dickkopf (Xenopus laevis) homolog 1 | |
| | /FL = gb: AF127563.1 gb: AF177394.1 | |
| | gb: NM_012242.1 | |
| FHL1: four and a half | gb: NM_001449.1 /DEF = Homo sapiens four | 201540_at |
| LIM domains 1 | and a half LIM domains 1 (FHL1), mRNA. | |
| (LOC2273) | /FEA = mRNA /GEN = FHL1 /PROD = four and a | |
| SEQ ID NOS: 8 | half LIM domains 1 /DB_XREF = gi: 4503720 | |
| (DNA) and 155 | /UG = Hs.239069 four and a half LIM domains 1 | |
| (amino acid) | /FL = gb: U29538.1 gb: U60115.1 | |
| | gb: NM_001449.1 | |
| SSR4: signal | gb: NM_006280.1 /DEF = Homo sapiens signal | 201004_at |
| sequence receptor, | sequence receptor, delta (translocon-associated | |
| delta (LOC6748) | protein delta) (SSR4), mRNA. /FEA = mRNA | |
| SEQ ID NOS: 9 | /GEN = SSR4 /PROD = signal sequence receptor, | |
| (DNA) and 156 | delta /DB_XREF = gi: 5454089 /UG = Hs.102135 | |
| (amino acid) | signal sequence receptor, delta (translocon- | |
| | associated protein delta) /FL = gb: BC003371.1 | |
| | gb: NM_006280.1 | |
| S100A9: S100 | gb: NM_002965.2 /DEF = Homo sapiens S100 | 203535_at |
| calcium-binding | calcium-binding protein A9 (calgranulin B) | |
| protein A9 | (S100A9), mRNA. /FEA = mRNA | |
| (LOC6280) | /GEN = S100A9 /PROD = S100 calcium-binding | |
| SEQ ID NOS: 10 | protein A9 /DB_XREF = gi: 9845520 | |
| (DNA) and 157 | /UG = Hs.112405 S100 calcium-binding protein | |
| (amino acid) | A9 (calgranulin B) /FL = gb: M26311.1 | |
| | gb: NM_002965.2 | |
| SFN: stratifin | Cluster Incl. X57348: H. sapiens mRNA (clone | 33322_i_at |
| (LOC2810) | 9112) /cds = (165, 911) /gb = X57348 /gi = 23939 | |
| SEQ ID NOS: 11 | /ug = Hs.184510 /len = 1407 | |
| (DNA) and 158 | | |
| (amino acid) | | |
| F2RL1: coagulation | Consensus includes gb: BE965369 /FEA = EST | 213506_at |
| factor II (thrombin) | /DB_XREF = gi: 11769659 | |
| receptor-like 1 | /DB_XREF = est: 601659282R1 | |
| precursor (LOC2150) | /CLONE = IMAGE: 3895653 /UG = Hs.168102 | |
| SEQ ID NOS: 12 | Human proteinase activated receptor-2 mRNA, | |
| (DNA) and 159 | 3UTR | |
| (amino acid) | | |
| SPUVE: protease, | gb: NM_007173.1 /DEF = Homo sapiens | 202458_at |
| serine, 23 precursor | protease, serine, 23 (SPUVE), mRNA. | |
| (LOC11098) | /FEA = mRNA /GEN = SPUVE /PROD = protease, | |
| SEQ ID NOS: 13 | serine, 23 /DB_XREF = gi: 6005881 | |
| (DNA) and 160 | /UG = Hs.325820 protease, serine, 23 | |
| (amino acid) | /FL = gb: AL136914.1 gb: BC001278.1 | |
| | gb: AF015287.1 gb: NM_007173.1 | |
| | gb: AF193611.1 | |
| AMIGO2: | Consensus includes gb: AC004010 | 222108_at |
| amphoterin induced | /DEF = Human BAC clone GS1-99H8 | |
| gene 2 (LOC347902) | /FEA = CDS /DB_XREF = gi: 2781385 | |
| SEQ ID NOS: 14 | /UG = Hs.121520 Human BAC clone GS1-99H8 | |
| (DNA) and 161 | | |
| (amino acid) | | |
| KRT7: keratin 7 | gb: BC002700.1 /DEF = Homo sapiens , Similar | 209016_s_at |
| (LOC3855) | to keratin 7, clone MGC: 3625, mRNA, | |
| SEQ ID NOS: 15 | complete cds. /FEA = mRNA /PROD = Similar to | |
| (DNA) and 162 | keratin 7 /DB_XREF = gi: 12803726 | |
| (amino acid) | /UG = Hs.23881 keratin 7 /FL = gb: BC002700.1 | |
| | gb: NM_005556.1 | |
| RPL13: ribosomal | Consensus includes gb: AW574664 /FEA = EST | 212191_x_at |
| protein L13 | /DB_XREF = gi: 7246203 /DB_XREF = est: UI- | |
| (LOC6137) | HF-BL0-abw-d-10-0-UI.s1 | |
| SEQ ID NOS: 16 | /CLONE = IMAGE: 3057859 /UG = Hs.180842 | |
| (DNA) and 163 | ribosomal protein L13 | |
| (amino acid) | | |
| AF1Q: AF1Q protein | gb: BC006471.1 /DEF = Homo sapiens , ALL1- | 211071_s_at |
| (LOC10962) | fused gene from chromosome 1q, clone | |
| SEQ ID NOS: 17 | MGC: 4013, mRNA, complete cds. | |
| (DNA) and 164 | /FEA = mRNA /PROD = ALL1-fused gene from | |
| (amino acid) | chromosome 1q /DB_XREF = gi: 13623686 | |
| | /FL = gb: BC006471.1 | |
| COL6A2: alpha 2 | gb: AY029208.1 /DEF = Homo sapiens type VI | 209156_s_at |
| type VI collagen | collagen alpha 2 chain precursor (COL6A2) | |
| isoform 2C2 | mRNA, complete cds, alternatively spliced. | |
| precursor (LOC1292) | /FEA = mRNA /GEN = COL6A2 /PROD = type VI | |
| SEQ ID NOS: 18 | collagen alpha 2 chain precursor | |
| (DNA) and 165 | /DB_XREF = gi: 13603393 /UG = Hs.159263 | |
| (amino acid) | collagen, type VI, alpha 2 /FL = gb: AY029208.1 | |
| COL6A1: collagen, | Consensus includes gb: AA292373 /FEA = EST | 213428_s_at |
| type VI, alpha 1 | /DB_XREF = gi: 1940353 | |
| precursor (LOC1291) | /DB_XREF = est: zt51a09.s1 | |
| SEQ ID NOS: 19 | /CLONE = IMAGE: 725848 /UG = Hs.108885 | |
| (DNA) and 166 | collagen, type VI, alpha 1 | |
| (amino acid) | | |
| SLC38A2: solute | gb: NM_018573.1 /DEF = Homo sapiens | 218041_x_at |
| carrier family 38, | hypothetical protein PRO1068 (PRO1068), | |
| member 2 | mRNA. /FEA = mRNA /GEN = PRO1068 | |
| (LOC54407) | /PROD = hypothetical protein PRO1068 | |
| SEQ ID NOS: 20 | /DB_XREF = gi: 8924006 /UG = Hs.321158 | |
| (DNA) and 167 | hypothetical protein PRO1068 | |
| (amino acid) | /FL = gb: AF116620.1 gb: NM_018573.1 | |
| PAPSS2: 3′- | gb: AF074331.1 /DEF = Homo sapiens PAPS | 203060_s_at |
| phosphoadenosine 5′- | synthetase-2 (PAPSS2) mRNA, complete cds. | |
| phosphosulfate | /FEA = mRNA /GEN = PAPSS2 /PROD = PAPS | |
| synthase 2 | synthetase-2 /DB_XREF = gi: 5052074 | |
| (LOC9060) | /UG = Hs.274230 3-phosphoadenosine 5- | |
| SEQ ID NOS: 21 | phosphosulfate synthase 2 /FL = gb: AF150754.2 | |
| (DNA) and 168 | gb: AF313907.1 gb: AF091242.1 | |
| (amino acid) | gb: NM_004670.1 gb: AF074331.1 | |
| | gb: AF173365.1 | |
| JAG1: jagged 1 | gb: U73936.1 /DEF = Homo sapiens Jagged 1 | 209099_x_at |
| precursor (LOC182) | (HJ1) mRNA, complete cds. /FEA = mRNA | |
| SEQ ID NOS: 22 | /GEN = HJ1 /PROD = Jagged 1 | |
| (DNA) and 169 | /DB_XREF = gi: 1695273 /UG = Hs.91143 jagged | |
| (amino acid) | 1 (Alagille syndrome) /FL = gb: U61276.1 | |
| | gb: U73936.1 gb: AF003837.1 gb: AF028593.1 | |
| | gb: NM_000214.1 | |
| RPS27L: ribosomal | gb: NM_015920.1 /DEF = Homo sapiens 40S | 218007_s_at |
| protein S27-like | ribosomal protein S27 isoform (LOC51065), | |
| protein (LOC51065) | mRNA. /FEA = mRNA /GEN = LOC51065 | |
| SEQ ID NOS: 23 | /PROD = 40S ribosomal protein S27 isoform | |
| (DNA) and 170 | /DB_XREF = gi: 7705705 /UG = Hs.108957 40S | |
| (amino acid) | ribosomal protein S27 isoform | |
| | /FL = gb: BC003667.1 gb: AF070668.1 | |
| | gb: NM_015920.1 | |
| PAM: peptidylglycine | gb: NM_000919.1 /DEF = Homo sapiens | 202336_s_at |
| alpha-amidating | peptidylglycine alpha-amidating | |
| monooxygenase | monooxygenase (PAM), mRNA. /FEA = mRNA | |
| isoform a, | /GEN = PAM /PROD = peptidylglycine alpha- | |
| preproprotein | amidating monooxygenase | |
| (LOC5066) | /DB_XREF = gi: 4505602 /UG = Hs.83920 | |
| SEQ ID NOS: 24 | peptidylglycine alpha-amidating | |
| (DNA) and 171 | monooxygenase /FL = gb: M37721.1 | |
| (amino acid) | gb: NM_000919.1 | |
| STAT1: signal | gb: NM_007315.1 /DEF = Homo sapiens signal | 200887_s_at |
| transducer and | transducer and activator of transcription 1, | |
| activator of | 91 kD (STAT1), mRNA. /FEA = mRNA | |
| transcription 1 | /GEN = STAT1 /PROD = signal transducer and | |
| isoform alpha | activator of transcription1, 91 kD | |
| (LOC6772) | /DB_XREF = gi: 6274551 /UG = Hs.21486 signal | |
| SEQ ID NOS: 25 | transducer and activator of transcription 1, | |
| (DNA) and 172 | 91 kD /FL = gb: M97935.1 gb: NM_007315.1 | |
| (amino acid) | | |
| CTSB: cathepsin B | gb: NM_001908.1 /DEF = Homo sapiens | 200839_s_at |
| preproprotein | cathepsin B (CTSB), mRNA. /FEA = mRNA | |
| (LOC1508) | /GEN = CTSB /PROD = cathepsin B | |
| SEQ ID NOS: 26 | /DB_XREF = gi: 4503138 /UG = Hs.297939 | |
| (DNA) and 173 | cathepsin B /FL = gb: M14221.1 gb: L16510.1 | |
| (amino acid) | gb: NM_001908.1 | |
| POLR2L: DNA | gb: BC005903.1 /DEF = Homo sapiens , | 211730_s_at |
| directed RNA | polymerase (RNA) II (DNA directed) | |
| polymerase II | polypeptide L (7.6 kD), clone MGC: 14494, | |
| polypeptide L | mRNA, complete cds. /FEA = mRNA | |
| (LOC5441) | /PROD = polymerase (RNA) II (DNA directed) | |
| SEQ ID NOS: 27 | polypeptide L(7.6 kD) /DB_XREF = gi: 13543491 | |
| (DNA) and 174 | /FL = gb: BC005903.1 | |
| (amino acid) | | |
| ETV1: ets variant | Consensus includes gb: BE881590 /FEA = EST | 221911_at |
| gene 1 (LOC2115) | /DB_XREF = gi: 10330366 | |
| SEQ ID NOS: 28 | /DB_XREF = est: 601490008F1 | |
| (DNA) and 175 | /CLONE = IMAGE: 3892465 /UG = Hs.10684 | |
| (amino acid) | Homo sapiens clone 24421 mRNA sequence | |
| KRT18: keratin 18 | gb: NM_000224.1 /DEF = Homo sapiens keratin | 201596_x_at |
| (LOC3875) | 18 (KRT18), mRNA. /FEA = mRNA | |
| SEQ ID NOS: 29 | /GEN = KRT18 /PROD = keratin 18 | |
| (DNA) and 176 | /DB_XREF = gi: 4557887 /UG = Hs.65114 keratin | |
| (amino acid) | 18 /FL = gb: BC000698.1 gb: BC000180.2 | |
| | gb: BC004253.1 gb: M26326.1 | |
| | gb: NM_000224.1 | |
| RPL29: ribosomal | Consensus includes gb: BF683426 /FEA = EST | 213969_x_at |
| protein L29 | /DB_XREF = gi: 11968834 | |
| (LOC6159) | /DB_XREF = est: 602139603F1 | |
| SEQ ID NOS: 30 | /CLONE = IMAGE: 4300777 /UG = Hs.183698 | |
| (DNA) and 177 | ribosomal protein L29 | |
| (amino acid) | | |
| PYGB: brain | gb: NM_002862.1 /DEF = Homo sapiens | 201481_s_at |
| glycogen | phosphorylase, glycogen; brain (PYGB), | |
| phosphorylase | nuclear gene encoding mitochondrial protein, | |
| (LOC5834) | mRNA. /FEA = mRNA /GEN = PYGB | |
| SEQ ID NOS: 31 | /PROD = phosphorylase, glycogen; brain | |
| (DNA) and 178 | /DB_XREF = gi: 4506350 /UG = Hs.75658 | |
| (amino acid) | phosphorylase, glycogen; brain | |
| | /FL = gb: U47025.1 gb: NM_002862.1 | |
| ALCAM: activated | Consensus includes gb: AA156721 /FEA = EST | 201952_at |
| leukocyte cell | /DB_XREF = gi: 1728335 | |
| adhesion molecule | /DB_XREF = est: zl18b04.s1 | |
| (LOC214) | /CLONE = IMAGE: 502255 /UG = Hs.10247 | |
| SEQ ID NOS: 32 | activated leucocyte cell adhesion molecule | |
| (DNA) and 179 | /FL = gb: NM_001627.1 gb: L38608.1 | |
| (amino acid) | | |
| CTGF: connective | gb: M92934.1 /DEF = Human connective tissue | 209101_at |
| tissue growth factor | growth factor, complete cds. /FEA = mRNA | |
| (LOC1490) | /PROD = connective tissue growth factor | |
| SEQ ID NOS: 33 | /DB_XREF = gi: 180923 /UG = Hs.75511 | |
| (DNA) and 180 | connective tissue growth factor | |
| (amino acid) | /FL = gb: M92934.1 gb: NM_001901.1 | |
| UCHL1: ubiquitin | gb: NM_004181.1 /DEF = Homo sapiens | 201387_s_at |
| carboxyl-terminal | ubiquitin carboxyl-terminal esterase L1 | |
| esterase L1 (ubiquitin | (ubiquitin thiolesterase) (UCHL1), mRNA. | |
| thiolesterase) | /FEA = mRNA /GEN = UCHL1 | |
| (LOC7345) | /PROD = ubiquitin carboxyl-terminal esterase | |
| SEQ ID NOS: 34 | L1(ubiquitin thiolesterase) | |
| (DNA) and 181 | /DB_XREF = gi: 4759283 /UG = Hs.76118 | |
| (amino acid) | ubiquitin carboxyl-terminal esterase L1 | |
| | (ubiquitin thiolesterase) /FL = gb: BC000332.1 | |
| | gb: BC005117.1 gb: NM_004181.1 | |
| C14orf78: | Consensus includes gb: AI935123 /FEA = EST | 212992_at |
| chromosome 14 open | /DB_XREF = gi: 5673993 | |
| reading frame 78 | /DB_XREF = est: wp13h09.x1 | |
| (LOC113146) | /CLONE = IMAGE: 2464769 /UG = Hs.57548 | |
| SEQ ID NOS: 35 | ESTs | |
| (DNA) and 182 | | |
| (amino acid) | | |
| PBEF: pre-B-cell | Consensus includes gb: BF575514 /FEA = EST | 217738_at |
| colony-enhancing | /DB_XREF = gi: 11649318 | |
| factor isoform a | /DB_XREF = est: 602133090F1 | |
| (LOC10135) | /CLONE = IMAGE: 4288079 /UG = Hs.239138 | |
| SEQ ID NOS: 36 | pre-B-cell colony-enhancing factor | |
| (DNA) and 183 | /FL = gb: U02020.1 gb: NM_005746.1 | |
| (amino acid) | | |
| GNG11: guanine | gb: NM_004126.1 /DEF = Homo sapiens guanine | 204115_at |
| nucleotide binding | nucleotide binding protein 11 (GNG11), | |
| protein (G protein), | mRNA. /FEA = mRNA /GEN = GNG11 | |
| gamma 11 | /PROD = guanine nucleotide binding protein 11 | |
| (LOC2791) | /DB_XREF = gi: 4758447 /UG = Hs.83381 | |
| SEQ ID NOS: 37 | guanine nucleotide binding protein 11 | |
| (DNA) and 184 | /FL = gb: NM_004126.1 gb: U31384.1 | |
| (amino acid) | | |
| SERPINE2: | Consensus includes gb: AL541302 /FEA = EST | 212190_at |
| plasminogen activator | /DB_XREF = gi: 12872241 | |
| inhibitor type 1, | /DB_XREF = est: AL541302 | |
| member 2 | /CLONE = CS0DE006YI10 (5 prime) | |
| (LOC5270) | /UG = Hs.21858 trinucleotide repeat containing 3 | |
| SEQ ID NOS: 38 | | |
| (DNA) and 185 | | |
| (amino acid) | | |
| PTTG1IP: pituitary | gb: NM_004339.2 /DEF = Homo sapiens | 200677_at |
| tumor-transforming | pituitary tumor-transforming 1 interacting | |
| gene 1 protein- | protein (PTTG1IP), mRNA. /FEA = mRNA | |
| interacting protein | /GEN = PTTG1IP /PROD = pituitary tumor- | |
| precursor (LOC754) | transforming protein1-interacting protein | |
| SEQ ID NOS: 39 | precursor /DB_XREF = gi: 11038670 | |
| (DNA) and 186 | /UG = Hs.111126 pituitary tumor-transforming 1 | |
| (amino acid) | interacting protein /FL = gb: NM_004339.2 | |
| | gb: BC000415.1 gb: AF149785.1 | |
| KRT19: keratin 19 | gb: NM_002276.1 /DEF = Homo sapiens keratin | 201650_at |
| (LOC3880) | 19 (KRT19), mRNA. /FEA = mRNA | |
| SEQ ID NOS: 40 | /GEN = KRT19 /PROD = keratin 19 | |
| (DNA) and 187 | /DB_XREF = gi: 4504916 /UG = Hs.182265 | |
| (amino acid) | keratin 19 /FL = gb: BC002539.1 | |
| | gb: NM_002276.1 | |
| SFN: stratifin | Cluster Incl. X57348: H. sapiens mRNA (clone | 33323_r_at |
| (LOC2810) | 9112) /cds = (165,911) /gb = X57348 /gi = 23939 | |
| SEQ ID NOS: 41 | /ug = Hs.184510 /len = 1407 | |
| (DNA) and 188 | | |
| (amino acid) | | |
| ICAM1: intercellular | Consensus includes gb: AI608725 /FEA = EST | 202637_s_at |
| adhesion molecule 1 | /DB_XREF = gi: 4617892 | |
| (LOC3383) | /DB_XREF = est: tw90b01.x1 | |
| SEQ ID NOS: 42 | /CLONE = IMAGE: 2266921 /UG = Hs.168383 | |
| (DNA) and 189 | intercellular adhesion molecule 1 (CD54), | |
| (amino acid) | human rhinovirus receptor /FL = gb: M24283.1 | |
| | gb: J03132.1 gb: NM_000201.1 | |
| SLC6A8: solute | gb: NM_005629.1 /DEF = Homo sapiens solute | 202219_at |
| carrier family 6 | carrier family 6 (neurotransmitter transporter, | |
| (neurotransmitter | creatine), member 8 (SLC6A8), mRNA. | |
| transporter, creatine), | /FEA = mRNA /GEN = SLC6A8 /PROD = solute | |
| member 8 | carrier family 6 (neurotransmittertransporter, | |
| (LOC6535) | creatine), member 8 /DB_XREF = gi: 5032096 | |
| SEQ ID NOS: 43 | /UG = Hs.187958 solute carrier family 6 | |
| (DNA) and 190 | (neurotransmitter transporter, creatine), member | |
| (amino acid) | 8 /FL = gb: L31409.1 gb: NM_005629.1 | |
| IL8: interleukin 8 | gb: AF043337.1 /DEF = Homo sapiens | 211506_s_at |
| (LOC3576) | interleukin 8 C-terminal variant (IL8) mRNA, | |
| SEQ ID NOS: 44 | complete cds. /FEA = mRNA /GEN = IL8 | |
| (DNA) and 191 | /PROD = interleukin 8 C-terminal variant | |
| (amino acid) | /DB_XREF = gi: 12641914 /UG = Hs.624 | |
| | interleukin 8 /FL = gb: AF043337.1 | |
| CSPG2: chondroitin | gb: NM_004385.1 /DEF = Homo sapiens | 204620_s_at |
| sulfate proteoglycan 2 | chondroitin sulfate proteoglycan 2 (versican) | |
| (versican) (LOC1462) | (CSPG2), mRNA. /FEA = mRNA | |
| SEQ ID NOS: 45 | /GEN = CSPG2 /PROD = chondroitin sulfate | |
| (DNA) and 192 | proteoglycan 2 (versican) | |
| (amino acid) | /DB_XREF = gi: 4758081 /UG = Hs.81800 | |
| | chondroitin sulfate proteoglycan 2 (versican) | |
| | /FL = gb: NM_004385.1 | |
| CTSC: cathepsin C | gb: NM_001814.1 /DEF = Homo sapiens | 201487_at |
| isoform a | cathepsin C (CTSC), mRNA. /FEA = mRNA | |
| preproprotein | /GEN = CTSC /PROD = cathepsin C | |
| (LOC1075) | /DB_XREF = gi: 4503140 /UG = Hs.10029 | |
| SEQ ID NOS: 46 | cathepsin C /FL = gb: NM_001814.1 | |
| (DNA) and 193 | | |
| (amino acid) | | |
| JTB: jumping | gb: BC004239.1 /DEF = Homo sapiens , jumping | 210927_x_at |
| translocation | translocation breakpoint, clone MGC: 10274, | |
| breakpoint | mRNA, complete cds. /FEA = mRNA | |
| (LOC10899) | /PROD = jumping translocation breakpoint | |
| SEQ ID NOS: 47 | /DB_XREF = gi: 13278986 /UG = Hs.323093 | |
| (DNA) and 194 | Homo sapiens , jumping translocation | |
| (amino acid) | breakpoint, clone MGC: 10274, mRNA, | |
| | complete cds /FL = gb: BC004239.1 | |
| KRT8: keratin 8 | gb: U76549.1 /DEF = Human cytokeratin 8 | 209008_x_at |
| (LOC3856) | mRNA, complete cds. /FEA = mRNA | |
| SEQ ID NOS: 48 | /PROD = cytokeratin 8 /DB_XREF = gi: 1673574 | |
| (DNA) and 195 | /UG = Hs.242463 keratin 8 /FL = gb: BC000654.1 | |
| (amino acid) | gb: U76549.1 gb: M34225.1 gb: M26324.1 | |
| | gb: NM_002273.1 | |
| UGDH: UDP-glucose | gb: NM_003359.1 /DEF = Homo sapiens UDP- | 203343_at |
| dehydrogenase | glucose dehydrogenase (UGDH), mRNA. | |
| (LOC7358) | /FEA = mRNA /GEN = UGDH /PROD = UDP- | |
| SEQ ID NOS: 49 | glucose dehydrogenase | |
| (DNA) and 196 | /DB_XREF = gi: 4507812 /UG = Hs.28309 UDP- | |
| (amino acid) | glucose dehydrogenase /FL = gb: AF061016.1 | |
| | gb: NM_003359.1 | |
| TXNIP: thioredoxin | Consensus includes gb: AA812232 /FEA = EST | 201008_s_at |
| interacting protein | /DB_XREF = gi: 2881843 | |
| (LOC10628) | /DB_XREF = est: ob84h09.s1 | |
| SEQ ID NOS: 50 | /CLONE = IMAGE: 1338113 /UG = Hs.179526 | |
| (DNA) and 197 | upregulated by 1,25-dihydroxyvitamin D-3 | |
| (amino acid) | /FL = gb: NM_006472.1 gb: S73591.1 | |
| CTSB: cathepsin B | gb: NM_001908.1 /DEF = Homo sapiens | 200838_at |
| preproprotein | cathepsin B (CTSB), mRNA. /FEA = mRNA | |
| (LOC1508) | /GEN = CTSB /PROD = cathepsin B | |
| SEQ ID NOS: 51 | /DB_XREF = gi: 4503138 /UG = Hs.297939 | |
| (DNA) and 198 | cathepsin B /FL = gb: M14221.1 gb: L16510.1 | |
| (amino acid) | gb: NM_001908.1 | |
| CSPG2: chondroitin | Consensus includes gb: BF218922 /FEA = EST | 221731_x_at |
| sulfate proteoglycan 2 | /DB_XREF = gi: 11112418 | |
| (versican) (LOC1462) | /DB_XREF = est: 601885091F1 | |
| SEQ ID NOS: 52 | /CLONE = IMAGE: 4103447 /UG = Hs.81800 | |
| (DNA) and 199 | chondroitin sulfate proteoglycan 2 (versican) | |
| (amino acid) | | |
| ANXA10: annexin | gb: AF196478.1 /DEF = Homo sapiens annexin | 210143_at |
| A10 (LOC11199) | 14 (ANX14) mRNA, complete cds. | |
| SEQ ID NOS: 53 | /FEA = mRNA /GEN = ANX14 /PROD = annexin | |
| (DNA) and 200 | 14 /DB_XREF = gi: 6274496 /UG = Hs.188401 | |
| (amino acid) | annexin A10 /FL = gb: AF196478.1 | |
| | gb: NM_007193.2 | |
| SAT: | gb: M55580.1 /DEF = Human | 210592_s_at |
| spermidine/spermine | spermidinespermine N1-acetyltransferase | |
| N1-acetyltransferase | mRNA, complete cds. /FEA = mRNA | |
| (LOC6303) | /GEN = spermidinespermine N1- | |
| SEQ ID NOS: 54 | acetyltransferase /PROD = spermidinespermine | |
| (DNA) and 201 | N1-acetyltransferase /DB_XREF = gi: 338335 | |
| (amino acid) | /UG = Hs.28491 spermidinespermine N1- | |
| | acetyltransferase /FL = gb: M55580.1 | |
| COL6A3: alpha 3 | gb: NM_004369.1 /DEF = Homo sapiens | 201438_at |
| type VI collagen | collagen, type VI, alpha 3 (COL6A3), mRNA. | |
| isoform 1 precursor | /FEA = mRNA /GEN = COL6A3 | |
| (LOC1293) | /PROD = collagen, type VI, alpha 3 | |
| SEQ ID NOS: 55 | /DB_XREF = gi: 4758027 /UG = Hs.80988 | |
| (DNA) and 202 | collagen, type VI, alpha 3 | |
| (amino acid) | /FL = gb: NM_004369.1 | |
| SPARC: secreted | gb: NM_003118.1 /DEF = Homo sapiens secreted | 200665_s_at |
| protein, acidic, | protein, acidic, cysteine-rich (osteonectin) | |
| cysteine-rich | (SPARC), mRNA. /FEA = mRNA | |
| (osteonectin) | /GEN = SPARC /PROD = secreted protein, acidic, | |
| (LOC6678) | cysteine-rich(osteonectin) | |
| SEQ ID NOS: 56 | /DB_XREF = gi: 4507170 /UG = Hs.111779 | |
| (DNA) and 203 | secreted protein, acidic, cysteine-rich | |
| (amino acid) | (osteonectin) /FL = gb: BC004974.1 gb: J03040.1 | |
| | gb: NM_003118.1 | |
| TXNIP: thioredoxin | gb: NM_006472.1 /DEF = Homo sapiens | 201010_s_at |
| interacting protein | upregulated by 1,25-dihydroxyvitamin D-3 | |
| (LOC10628) | (VDUP1), mRNA. /FEA = mRNA | |
| SEQ ID NOS: 57 | /GEN = VDUP1 /PROD = upregulated by 1,25- | |
| (DNA) and 204 | dihydroxyvitamin D-3 /DB_XREF = gi: 5454161 | |
| (amino acid) | /UG = Hs.179526 upregulated by 1,25- | |
| | dihydroxyvitamin D-3 /FL = gb: NM_006472.1 | |
| | gb: S73591.1 | |
| MDK: midkine | gb: M69148.1 /DEF = Human midkine mRNA, | 209035_at |
| (neurite growth- | complete cds. /FEA = mRNA /GEN = hMK-1 | |
| promoting factor 2) | /PROD = midkine /DB_XREF = gi: 182650 | |
| (LOC4192) | /UG = Hs.82045 midkine (neurite growth- | |
| SEQ ID NOS: 58 | promoting factor 2) /FL = gb: M69148.1 | |
| (DNA) and 205 | gb: NM_002391.1 | |
| (amino acid) | | |
| TXNRD1: | gb: NM_003330.1 /DEF = Homo sapiens | 201266_at |
| thioredoxin reductase | thioredoxin reductase 1 (TXNRD1), mRNA. | |
| 1 (LOC7296) | /FEA = mRNA /GEN = TXNRD1 | |
| SEQ ID NOS: 59 | /PROD = thioredoxin reductase 1 | |
| (DNA) and 206 | /DB_XREF = gi: 4507746 /UG = Hs.13046 | |
| (amino acid) | thioredoxin reductase 1 /FL = gb: D88687.1 | |
| | gb: AF077367.1 gb: NM_003330.1 | |
| | gb: AF208018.1 | |
| ARHD: ras homolog | gb: BC001338.1 /DEF = Homo sapiens , ras | 209885_at |
| D (LOC29984) | homolog gene family, member, clone | |
| SEQ ID NOS: 60 | MGC: 5612, mRNA, complete cds. | |
| (DNA) and 207 | /FEA = mRNA /PROD = ras homolog gene | |
| (amino acid) | family, member /DB_XREF = gi: 12654980 | |
| | /UG = Hs.15114 ras homolog gene family, | |
| | member /FL = gb: BC001338.1 gb: NM_014578.1 | |
| PSPHL: | gb: NM_003832.1 /DEF = Homo sapiens | 205048_s_at |
| phosphoserine | phosphoserine phosphatase-like (PSPHL), | |
| phosphatase-like | mRNA. /FEA = mRNA /GEN = PSPHL | |
| (LOC8781) | /PROD = L-3-phosphoserine phosphatase | |
| SEQ ID NOS: 61 | homolog /DB_XREF = gi: 4502934 | |
| (DNA) and 208 | /UG = Hs.76845 phosphoserine phosphatase-like | |
| (amino acid) | /FL = gb: NM_003832.1 | |
| RAB25: RAB25 | gb: NM_020387.1 /DEF = Homo sapiens CATX- | 218186_at |
| (LOC57111) | 8 protein (CATX-8), mRNA. /FEA = mRNA | |
| SEQ ID NOS: 62 | /GEN = CATX-8 /PROD = CATX-8 protein | |
| (DNA) and 209 | /DB_XREF = gi: 9966860 /UG = Hs.150826 | |
| (amino acid) | CATX-8 protein /FL = gb: AF083124.1 | |
| | gb: NM_020387.1 | |
| SPINT1: hepatocyte | gb: NM_003710.1 /DEF = Homo sapiens serine | 202826_at |
| growth factor | protease inhibitor, Kunitz type 1 (SPINT1), | |
| activator inhibitor 1 | mRNA. /FEA = mRNA /GEN = SPINT1 | |
| isoform 2 precursor | /PROD = hepatocyte growth factor activator | |
| (LOC6692) | inhibitorprecursor /DB_XREF = gi: 4504328 | |
| SEQ ID NOS: 63 | /UG = Hs.233950 serine protease inhibitor, | |
| (DNA) and 210 | Kunitz type 1 /FL: = gb: BC004140.1 | |
| (amino acid) | gb: AB000095.1 gb: NM_003710.1 | |
| SPINT2: serine | gb: AF027205.1 /DEF = Homo sapiens Kunitz- | 210715_s_at |
| protease inhibitor, | type protease inhibitor (kop) mRNA, complete | |
| Kunitz type, 2 | cds. /FEA = mRNA /GEN = kop /PROD = Kunitz- | |
| (LOC10653) | type protease inhibitor /DB_XREF = gi: 2598967 | |
| SEQ ID NOS: 64 | /UG = Hs.31439 serine protease inhibitor, Kunitz | |
| (DNA) and 211 | type, 2 /FL = gb: AF027205.1 | |
| (amino acid) | | |
| EMP3: epithelial | gb: NM_001425.1 /DEF = Homo sapiens | 203729_at |
| membrane protein 3 | epithelial membrane protein 3 (EMP3), mRNA. | |
| (LOC2014) | /FEA = mRNA /GEN = EMP3 /PROD = epithelial | |
| SEQ ID NOS: 65 | membrane protein 3 /DB_XREF = gi: 4503562 | |
| (DNA) and 212 | /UG = Hs.9999 epithelial membrane protein 3 | |
| (amino acid) | /FL = gb: U52101.1 gb: U87947.1 | |
| | gb: NM_001425.1 | |
| TENS1: tensin-like | gb: NM_022748.1 /DEF = Homo sapiens | 217853_at |
| SH2 domain- | hypothetical protein FLJ13732 similar to tensin | |
| containing 1 | (FLJ13732), mRNA. /FEA = mRNA | |
| (LOC64759) | /GEN = FLJ13732 /PROD = hypothetical protein | |
| SEQ ID NOS: 66 | FLJ13732 similar to tensin | |
| (DNA) and 213 | /DB_XREF = gi: 12232408 /UG = Hs.12210 | |
| (amino acid) | hypothetical protein FLJ13732 similar to tensin | |
| | /FL = gb: NM_022748.1 | |
| HIF1A: hypoxia- | gb: NM_001530.1 /DEF = Homo sapiens | 200989_at |
| inducible factor 1, | hypoxia-inducible factor 1, alpha subunit (basic | |
| alpha subunit isoform | helix-loop-helix transcription factor) (HIF1A), | |
| 1 (LOC3091) | mRNA. /FEA = mRNA /GEN = HIF1A | |
| SEQ ID NOS: 67 | /PROD = hypoxia-inducible factor 1, alpha | |
| (DNA) and 214 | subunit (basichelix-loop-helix transcription | |
| (amino acid) | factor) /DB_XREF = gi: 4504384 | |
| | /UG = Hs.197540 hypoxia-inducible factor 1, | |
| | alpha subunit (basic helix-loop-helix | |
| | transcription factor) /FL = gb: U29165.1 | |
| | gb: AF304431.1 gb: NM_001530.1 | |
| | gb: AF207601.1 gb: AF207602.1 gb: U22431.1 | |
| ST14: matriptase | gb: NM_021978.1 /DEF = Homo sapiens | 202005_at |
| (LOC6768) | suppression of tumorigenicity 14 (colon | |
| SEQ ID NOS: 68 | carcinoma, matriptase, epithin) (ST14), mRNA. | |
| (DNA) and 215 | /FEA = mRNA /GEN = ST14 | |
| (amino acid) | /PROD = suppression of tumorigenicity 14 | |
| | (coloncarcinoma, matriptase, epithin) | |
| | /DB_XREF = gi: 11415039 /UG = Hs.56937 | |
| | suppression of tumorigenicity 14 (colon | |
| | carcinoma, matriptase, epithin) | |
| | /FL = gb: AF057145.1 gb: NM_021978.1 | |
| | gb: AB030036.1 gb: AF133086.1 | |
| | gb: AF118224.2 | |
| STK17A: | Consensus includes gb: AW194730 /FEA = EST | 202693_s_at |
| serine/threonine | /DB_XREF = gi: 6473630 | |
| kinase 17a | /DB_XREF = est: xn43d11.x1 | |
| (apoptosis-inducing) | /CLONE = IMAGE: 2696469 /UG = Hs.9075 | |
| (LOC9263) | serinethreonine kinase 17a (apoptosis-inducing) | |
| SEQ ID NOS: 69 | /FL = gb: AB011420.1 gb: NM_004760.1 | |
| (DNA) and 216 | | |
| (amino acid) | | |
| SH3YL1: | gb: NM_015677.1 /DEF = Homo sapiens | 204019_s_at |
| hypothetical protein | hypothetical protein (DKFZP586F1318), | |
| DKFZP586F1318 | mRNA. /FEA = mRNA | |
| (LOC26751) | /GEN = DKFZP586F1318 /PROD = hypothetical | |
| SEQ ID NOS: 70 | protein /DB_XREF = gi: 7661669 /UG = Hs.25213 | |
| (DNA) and 217 | hypothetical protein /FL = gb: NM_015677.1 | |
| (amino acid) | | |
| EXT1: exostoses | gb: NM_000127.1 /DEF = Homo sapiens | 201995_at |
| (multiple) 1 | exostoses (multiple) 1 (EXT1), mRNA. | |
| (LOC2131) | /FEA = mRNA /GEN = EXT1 /PROD = exostoses | |
| SEQ ID NOS: 71 | (multiple) 1 /DB_XREF = gi: 4557570 | |
| (DNA) and 218 | /UG = Hs.184161 exostoses (multiple) 1 | |
| (amino acid) | /FL = gb: BC001174.1 gb: NM_000127.1 | |
| GALNT7: | gb: NM_017423.1 /DEF = Homo sapiens UDP- | 218313_s_at |
| polypeptide N- | N-acetyl-alpha-D-galactosamine: polypeptide | |
| acetylgalactosaminyltransferase 7 | N-acetylgalactosaminyltransferase 7 (GalNAc- | |
| (LOC51809) | T7) (GALNT7), mRNA. /FEA = mRNA | |
| SEQ ID NOS: 72 | /GEN = GALNT7 /PROD = polypeptide N- | |
| (DNA) and 219 | acetylgalactosaminyltransferase 7 | |
| (amino acid) | /DB_XREF = gi: 8393408 /UG = Hs.246315 UDP- | |
| | N-acetyl-alpha-D-galactosamine: polypeptide | |
| | N-acetylgalactosaminyltransferase 7 (GalNAc- | |
| | T7) /FL = gb: NM_017423.1 | |
| SDC1: syndecan 1 | gb: NM_002997.1 /DEF = Homo sapiens | 201287_s_at |
| (LOC6382) | syndecan 1 (SDC1), mRNA. /FEA = mRNA | |
| SEQ ID NOS: 73 | /GEN = SDC1 /PROD = syndecan 1 | |
| (DNA) and 220 | /DB_XREF = gi: 4506858 /UG = Hs.82109 | |
| (amino acid) | syndecan 1 /FL = gb: J05392.1 gb: NM_002997.1 | |
| ITGAV: integrin, | Consensus includes gb: AI093579 /FEA = EST | 202351_at |
| alpha V (vitronectin | /DB_XREF = gi: 3432555 | |
| receptor, alpha | /DB_XREF = est: qb15g06.x1 | |
| polypeptide, antigen | /CLONE = IMAGE: 1696378 /UG = Hs.295726 | |
| CD51) (LOC3685) | integrin, alpha V (vitronectin receptor, alpha | |
| SEQ ID NOS: 74 | polypeptide, antigen CD51) /FL = gb: M14648.1 | |
| (DNA) and 221 | gb: NM_002210.1 | |
| (amino acid) | | |
| ANXA6: annexin VI | gb: NM_001155.2 /DEF = Homo sapiens annexin | 200982_s_at |
| isoform 1 (LOC309) | A6 (ANXA6), transcript variant 1, mRNA. | |
| SEQ ID NOS: 75 | /FEA = mRNA /GEN = ANXA6 /PROD = annexin | |
| (DNA) and 222 | VI isoform 1 /DB_XREF = gi: 4809274 | |
| (amino acid) | /UG = Hs.118796 annexin A6 /FL = gb: J03578.1 | |
| | gb: D00510.1 gb: NM_001155.2 | |
| PDGFC: platelet- | gb: NM_016205.1 /DEF = Homo sapiens platelet | 218718_at |
| derived growth factor | derived growth factor C (PDGFC), mRNA. | |
| C precursor | /FEA = mRNA /GEN = PDGFC | |
| (LOC56034) | /PROD = secretory growth factor-like protein | |
| SEQ ID NOS: 76 | fallotein /DB_XREF = gi: 9994186 | |
| (DNA) and 223 | /UG = Hs.43080 platelet derived growth factor C | |
| (amino acid) | /FL = gb: AF091434.1 gb: AF244813.1 | |
| | gb: AB033831.1 gb: NM_016205.1 | |
| FLNA: filamin 1 | Consensus includes gb: AI625550 /FEA = EST | 214752_x_at |
| (actin-binding | /DB_XREF = gi: 4650481 | |
| protein-280) | /DB_XREF = est: ty57d06.x1 | |
| (LOC2316) | /CLONE = IMAGE: 2283179 /UG = Hs.195464 | |
| SEQ ID NOS: 77 | filamin A, alpha (actin-binding protein-280) | |
| (DNA) and 224 | | |
| (amino acid) | | |
| FLNA: filamin 1 | Consensus includes gb: AW051856 /FEA = EST | 213746_s_at |
| (actin-binding | /DB_XREF = gi: 5914215 | |
| protein-280) | /DB_XREF = est: wz04a05.x1 | |
| (LOC2316) | /CLONE = IMAGE: 2557040 /UG = Hs.195464 | |
| SEQ ID NOS: 78 | filamin A, alpha (actin-binding protein-280) | |
| (DNA) and 225 | | |
| (amino acid) | | |
| TUBA3: tubulin, | gb: AF141347.1 /DEF = Homo sapiens hum-a- | 209118_s_at |
| alpha 3 (LOC7846) | tub2 alpha-tubulin mRNA, complete cds. | |
| SEQ ID NOS: 79 | /FEA = mRNA /PROD = alpha-tubulin | |
| (DNA) and 226 | /DB_XREF = gi: 4929133 /UG = Hs.272897 | |
| (amino acid) | Tubulin, alpha, brain-specific | |
| | /FL = gb: AF141347.1 gb: NM_006009.1 | |
| LOXL2: lysyl | gb: NM_002318.1 /DEF = Homo sapiens lysyl | 202998_s_at |
| oxidase-like 2 | oxidase-like 2 (LOXL2), mRNA. /FEA = mRNA | |
| (LOC4017) | /GEN = LOXL2 /PROD = lysyl oxidase-like 2 | |
| SEQ ID NOS: 80 | /DB_XREF = gi: 4505010 /UG = Hs.83354 lysyl | |
| (DNA) and 227 | oxidase-like 2 /FL = gb: BC000594.1 | |
| (amino acid) | gb: U89942.1 gb: NM_002318.1 gb: AF117949.1 | |
| CYR61: cysteine- | gb: AF003114.1 /DEF = Homo sapiens CYR61 | 210764_s_at |
| rich, angiogenic | mRNA, complete cds. /FEA = mRNA | |
| inducer, 61 | /GEN = CYR61 /DB_XREF = gi: 6649848 | |
| (LOC3491) | /UG = Hs.8867 cysteine-rich, angiogenic | |
| SEQ ID NOS: 81 | inducer, 61 /FL = gb: AF003114.1 | |
| (DNA) and 228 | | |
| (amino acid) | | |
| GALNT3: | Consensus includes gb: BF063271 /FEA = EST | 203397_s_at |
| polypeptide N- | /DB_XREF = gi: 10822181 | |
| acetylgalactosaminyltransferase 3 | /DB_XREF = est: 7h87d05.x1 | |
| (LOC2591) | /CLONE = IMAGE: 3322953 /UG = Hs.278611 | |
| SEQ ID NOS: 82 | UDP-N-acetyl-alpha-D- | |
| (DNA) and 229 | galactosamine: polypeptide N- | |
| (amino acid) | acetylgalactosaminyltransferase 3 (GalNAc-T3) | |
| | /FL = gb: NM_004482.2 | |
| MAP1B: | Consensus includes gb: AL523076 /FEA = EST | 212233_at |
| microtubule- | /DB_XREF = gi: 12786569 | |
| associated protein 1B | /DB_XREF = est: AL523076 | |
| isoform 1 (LOC4131) | /CLONE = CS0DC001YI12 (3 prime) | |
| SEQ ID NOS: 83 | /UG = Hs.82503 H. sapiens mRNA for 3UTR of | |
| (DNA) and 230 | unknown protein | |
| (amino acid) | | |
| TUBB-5: tubulin | gb: BC002654.1 /DEF = Homo sapiens , Similar | 209191_at |
| beta-5 (LOC84617) | to tubulin, beta, 4, clone MGC: 4083, mRNA, | |
| SEQ ID NOS: 84 | complete cds. /FEA = mRNA /PROD = Similar to | |
| (DNA) and 231 | tubulin, beta, 4 /DB_XREF = gi: 12803638 | |
| (amino acid) | /UG = Hs.274398 Homo sapiens , Similar to | |
| | tubulin, beta, 4, clone MGC: 4083, mRNA, | |
| | complete cds /FL = gb: BC002654.1 | |
| TYMS: thymidylate | gb: NM_001071.1 /DEF = Homo sapiens | 202589_at |
| synthetase | thymidylate synthetase (TYMS), mRNA. | |
| (LOC7298) | /FEA = mRNA /GEN = TYMS | |
| SEQ ID NOS: 85 | /PROD = thymidylate synthetase | |
| (DNA) and 232 | /DB_XREF = gi: 4507750 /UG = Hs.82962 | |
| (amino acid) | thymidylate synthetase /FL = gb: BC002567.1 | |
| | gb: NM_001071.1 | |
| IFI16: interferon, | gb: NM_005531.1 /DEF = Homo sapiens | 206332_s_at |
| gamma-inducible | interferon, gamma-inducible protein 16 (IFI16), | |
| protein 16 | mRNA. /FEA = mRNA /GEN = IFI16 | |
| (LOC3428) | /PROD = interferon, gamma-inducible protein 16 | |
| SEQ ID NOS: 86 | /DB_XREF = gi: 5031778 /UG = Hs.155530 | |
| (DNA) and 233 | interferon, gamma-inducible protein 16 | |
| (amino acid) | /FL = gb: M63838.1 gb: NM_005531.1 | |
| GRB10: growth | gb: D86962.1 /DEF = Human mRNA for | 209409_at |
| factor receptor-bound | KIAA0207 gene, complete cds. /FEA = mRNA | |
| protein 10 | /GEN = KIAA0207 /DB_XREF = gi: 1503997 | |
| (LOC2887) | /UG = Hs.81875 growth factor receptor-bound | |
| SEQ ID NOS: 87 | protein 10 /FL = gb: D86962.1 gb: AF000017.1 | |
| (DNA) and 234 | | |
| (amino acid) | | |
| FLNA: filamin 1 | gb: NM_001456.1 /DEF = Homo sapiens filamin | 200859_x_at |
| (actin-binding | A, alpha (actin-binding protein-280) (FLNA), | |
| protein-280) | mRNA. /FEA = mRNA /GEN = FLNA | |
| (LOC2316) | /PROD = filamin 1 (actin-binding protein-280) | |
| SEQ ID NOS: 88 | /DB_XREF = gi: 4503744 /UG = Hs.195464 | |
| (DNA) and 235 | filamin A, alpha (actin-binding protein-280) | |
| (amino acid) | /FL = gb: NM_001456.1 | |
| TNC: tenascin C | gb: NM_002160.1 /DEF = Homo sapiens | 201645_at |
| (hexabrachion) | hexabrachion (tenascin C, cytotactin) (HXB), | |
| (LOC3371) | mRNA. /FEA = mRNA /GEN = HXB | |
| SEQ ID NOS: 89 | /PROD = hexabrachion (tenascin C, cytotactin) | |
| (DNA) and 236 | /DB_XREF = gi: 4504548 /UG = Hs.289114 | |
| (amino acid) | hexabrachion (tenascin C, cytotactin) | |
| | /FL = gb: M55618.1 gb: NM_002160.1 | |
| SLC26A2: sulfate | Consensus includes gb: AI025519 /FEA = EST | 205097_at |
| anion transporter 1 | /DB_XREF = gi: 3241132 | |
| (LOC1836) | /DB_XREF = est: ov75c04.x1 | |
| SEQ ID NOS: 90 | /CLONE = IMAGE: 1643142 /UG = Hs.29981 | |
| (DNA) and 237 | solute carrier family 26 (sulfate transporter), | |
| (amino acid) | member 2 /FL = gb: NM_000112.1 gb: U14528.1 | |
| KIAA0746: | Consensus includes gb: AB018289.1 | 212314_at |
| KIAA0746 protein | /DEF = Homo sapiens mRNA for KIAA0746 | |
| (LOC23231) | protein, partial cds. /FEA = mRNA | |
| SEQ ID NOS: 91 | /GEN = KIAA0746 /PROD = KIAA0746 protein | |
| (DNA) and 238 | /DB_XREF = gi: 3882212 /UG = Hs.49500 | |
| (amino acid) | KIAA0746 protein | |
| LAMP1: lysosomal- | gb: NM_005561.2 /DEF = Homo sapiens | 201553_s_at |
| associated membrane | lysosomal-associated membrane protein 1 | |
| protein 1 (LOC3916) | (LAMP1), mRNA. /FEA = mRNA | |
| SEQ ID NOS: 92 | /GEN = LAMP1 /PROD = lysosomal-associated | |
| (DNA) and 239 | membrane protein 1 /DB_XREF = gi: 7669500 | |
| (amino acid) | /UG = Hs.150101 lysosomal-associated | |
| | membrane protein 1 /FL = gb: J04182.1 | |
| | gb: J03263.1 gb: NM_005561.2 | |
| DPYSL2: | gb: NM_001386.1 /DEF = Homo sapiens | 200762_at |
| dihydropyrimidinase- | dihydropyrimidinase-like 2 (DPYSL2), mRNA. | |
| like 2 (LOC1808) | /FEA = mRNA /GEN = DPYSL2 | |
| SEQ ID NOS: 93 | /PROD = dihydropyrimidinase-like 2 | |
| (DNA) and 240 | /DB_XREF = gi: 4503376 /UG = Hs.173381 | |
| (amino acid) | dihydropyrimidinase-like 2 /FL = gb: U17279.1 | |
| | gb: D78013.1 gb: U97105.1 gb: NM_001386.1 | |
| IFI16: interferon, | gb: AF208043.1 /DEF = Homo sapiens IFI16b | 208966_x_at |
| gamma-inducible | (IFI16b) mRNA, complete cds. /FEA = mRNA | |
| protein 16 | /GEN = IFI16b /PROD = IFI16b | |
| (LOC3428) | /DB_XREF = gi: 6644296 /UG = Hs.155530 | |
| SEQ ID NOS: 94 | interferon, gamma-inducible protein 16 | |
| (DNA) and 241 | /FL = gb: AF208043.1 | |
| (amino acid) | | |
| KPNB2: karyopherin | Consensus includes gb: AI307759 /FEA = EST | 221829_s_at |
| beta 2 (LOC3842) | /DB_XREF = gi: 4002363 | |
| SEQ ID NOS: 95 | /DB_XREF = est: tb24g08.x1 | |
| (DNA) and 242 | /CLONE = IMAGE: 2055326 /UG = Hs.168075 | |
| (amino acid) | karyopherin (importin) beta 2 | |
| PRNP: prion protein | gb: NM_000311.1 /DEF = Homo sapiens prion | 201300_s_at |
| preproprotein | protein (p27-30) (Creutzfeld-Jakob disease, | |
| (LOC5621) | Gerstmann-Strausler-Scheinker syndrome, fatal | |
| SEQ ID NOS: 96 | familial insomnia) (PRNP), mRNA. | |
| (DNA) and 243 | /FEA = mRNA /GEN = PRNP /PROD = prion | |
| (amino acid) | protein /DB_XREF = gi: 4506112 /UG = Hs.74621 | |
| | prion protein (p27-30) (Creutzfeld-Jakob | |
| | disease, Gerstmann-Strausler-Scheinker | |
| | syndrome, fatal familial insomnia) | |
| | /FL = gb: AY008282.1 gb: M13899.1 | |
| | gb: NM_000311.1 | |
| RAI14: retinoic acid | gb: NM_015577.1 /DEF = Homo sapiens novel | 202052_s_at |
| induced 14 | retinal pigment epithelial gene (NORPEG), | |
| (LOC26064) | mRNA. /FEA = mRNA /GEN = NORPEG | |
| SEQ ID NOS: 97 | /PROD = DKFZP564G013 protein | |
| (DNA) and 244 | /DB_XREF = gi: 13470085 /UG = Hs.15165 novel | |
| (amino acid) | retinal pigment epithelial gene | |
| | /FL = gb: NM_015577.1 gb: AF155135.1 | |
| JAG1: jagged 1 | gb: U61276.1 /DEF = Human transmembrane | 209098_s_at |
| precursor (LOC182) | protein Jagged 1 (HJ1) mRNA, complete cds. | |
| SEQ ID NOS: 98 | /FEA = mRNA /GEN = HJ1 | |
| (DNA) and 245 | /PROD = transmembrane protein Jagged 1 | |
| (amino acid) | /DB_XREF = gi: 1438936 /UG = Hs.91143 jagged | |
| | 1 (Alagille syndrome) /FL = gb: U61276.1 | |
| | gb: U73936.1 gb: AF003837.1 gb: AF028593.1 | |
| | gb: NM_000214.1 | |
| CLIC4: chloride | gb: NM_013943.1 /DEF = Homo sapiens chloride | 201560_at |
| intracellular channel | intracellular channel 4 (CLIC4), mRNA. | |
| 4 (LOC25932) | /FEA = mRNA /GEN = CLIC4 /PROD = chloride | |
| SEQ ID NOS: 99 | intracellular channel 4 /DB_XREF = gi: 7330334 | |
| (DNA) and 246 | /UG = Hs.25035 chloride intracellular channel 4 | |
| (amino acid) | /FL = gb: AF109196.1 gb: AF097330.1 | |
| | gb: AL117424.1 gb: NM_013943.1 | |
| TP53I3: tumor | gb: BC000474.1 /DEF = Homo sapiens , quinone | 210609_s_at |
| protein p53 inducible | oxidoreductase homolog, clone MGC: 8642, | |
| protein 3 (LOC9540) | mRNA, complete cds. /FEA = mRNA | |
| SEQ ID NOS: 100 | /PROD = quinone oxidoreductase homolog | |
| (DNA) and 247 | /DB_XREF = gi: 12653408 /UG = Hs.50649 | |
| (amino acid) | quinone oxidoreductase homolog | |
| | /FL = gb: BC000474.1 | |
| EFA6R: ADP- | Consensus includes gb: AW117368 /FEA = EST | 203354_s_at |
| ribosylation factor | /DB_XREF = gi: 6085952 | |
| guanine nucleotide | /DB_XREF = est: xd88h01.x1 | |
| factor 6 (LOC23362) | /CLONE = IMAGE: 2604721 /UG = Hs.6763 | |
| SEQ ID NOS: 101 | KIAA0942 protein /FL = gb: AF243495.2 | |
| (DNA) and 248 | gb: NM_015310.1 | |
| (amino acid) | | |
| JUP: junction | gb: NM_021991.1 /DEF = Homo sapiens junction | 201015_s_at |
| plakoglobin | plakoglobin (JUP), transcript variant 2, mRNA. | |
| (LOC3728) | /FEA = mRNA /GEN = JUP /PROD = junction | |
| SEQ ID NOS: 102 | plakoglobin, isoform 1 | |
| (DNA) and 249 | /DB_XREF = gi: 12056467 /UG = Hs.2340 | |
| (amino acid) | junction plakoglobin /FL = gb: NM_021991.1 | |
| | gb: BC000441.1 | |
| PAPSS2: 3′- | gb: NM_004670.1 /DEF = Homo sapiens 3- | 203059_s_at |
| phosphoadenosine 5′- | phosphoadenosine 5-phosphosulfate synthase 2 | |
| phosphosulfate | (PAPSS2), mRNA. /FEA = mRNA | |
| synthase 2 | /GEN = PAPSS2 /PROD = 3-prime- | |
| (LOC9060) | phosphoadenosine 5-prime- | |
| SEQ ID NOS: 103 | phosphosulfatesynthase 2 | |
| (DNA) and 250 | /DB_XREF = gi: 4758879 /UG = Hs.274230 3- | |
| (amino acid) | phosphoadenosine 5-phosphosulfate synthase 2 | |
| | /FL = gb: AF150754.2 gb: AF313907.1 | |
| | gb: AF091242.1 gb: NM_004670.1 | |
| | gb: AF074331.1 gb: AF173365.1 | |
| DKK3: dickkopf | Consensus includes gb: AU148057 /FEA = EST | 214247_s_at |
| homolog 3 | /DB_XREF = gi: 11009578 | |
| (LOC27122) | /DB_XREF = est: AU148057 | |
| SEQ ID NOS: 104 | /CLONE = MAMMA1002489 /UG = Hs.278503 | |
| (DNA) and 251 | regulated in glioma | |
| (amino acid) | | |
| JAG1: jagged 1 | Consensus includes gb: U77914.1 /DEF = Human | 216268_s_at |
| precursor (LOC182) | soluble protein Jagged mRNA, partial cds. | |
| SEQ ID NOS: 105 | /FEA = mRNA /PROD = soluble protein Jagged | |
| (DNA) and 252 | /DB_XREF = gi: 1684889 /UG = Hs.91143 jagged | |
| (amino acid) | 1 (Alagille syndrome) | |
| CALD1: caldesmon 1 | Consensus includes gb: AL583520 /FEA = EST | 212077_at |
| isoform 3 (LOC800) | /DB_XREF = gi: 12952562 | |
| SEQ ID NOS: 106 | /DB_XREF = est: AL583520 | |
| (DNA) and 253 | /CLONE = CS0DC024YE13 (5 prime) | |
| (amino acid) | /UG = Hs.182183 Homo sapiens mRNA for | |
| | caldesmon, 3 UTR | |
| DPYSL3: | Consensus includes gb: W72516 /FEA = EST | 201430_s_at |
| dihydropyrimidinase- | /DB_XREF = gi: 1382173 | |
| like 3 (LOC1809) | /DB_XREF = est: zd64g05.s1 | |
| SEQ ID NOS: 107 | /CLONE = IMAGE: 345464 /UG = Hs.74566 | |
| (DNA) and 254 | dihydropyrimidinase-like 3 /FL = gb: D78014.1 | |
| (amino acid) | gb: NM_001387.1 | |
| PMP22: peripheral | gb: L03203.1 /DEF = Human peripheral myelin | 210139_s_at |
| myelin protein 22 | protein 22 (GAS3) mRNA, complete cds. | |
| (LOC5376) | /FEA = mRNA /GEN = GAS3 /PROD = peripheral | |
| SEQ ID NOS: 108 | myelin protein 22 /DB_XREF = gi: 182984 | |
| (DNA) and 255 | /UG = Hs.103724 peripheral myelin protein 22 | |
| (amino acid) | /FL = gb: L03203.1 | |
| ALCAM: activated | Consensus includes gb: BF242905 /FEA = EST | 201951_at |
| leukocyte cell | /DB_XREF = gi: 11156833 | |
| adhesion molecule | /DB_XREF = est: 601877949F1 | |
| (LOC214) | /CLONE = IMAGE: 4106028 /UG = Hs.10247 | |
| SEQ ID NOS: 109 | activated leucocyte cell adhesion molecule | |
| (DNA) and 256 | /FL = gb: NM_001627.1 gb: L38608.1 | |
| (amino acid) | | |
| PAPSS2: 3′- | Consensus includes gb: AW299958 /FEA = EST | 203058_s_at |
| phosphoadenosine 5′- | /DB_XREF = gi: 6709635 | |
| phosphosulfate | /DB_XREF = est: xs44g05.x1 | |
| synthase 2 | /CLONE = IMAGE: 2772536 /UG = Hs.274230 3- | |
| (LOC9060) | phosphoadenosine 5-phosphosulfate synthase 2 | |
| SEQ ID NOS: 110 | /FL = gb: AF150754.2 gb: AF313907.1 | |
| (DNA) and 257 | gb: AF091242.1 gb: NM_004670.1 | |
| (amino acid) | gb: AF074331.1 gb: AF173365.1 | |
| KPNB2: karyopherin | gb: NM_002270.1 /DEF = Homo sapiens | 207657_x_at |
| beta 2 (LOC3842) | karyopherin (importin) beta 2 (KPNB2), | |
| SEQ ID NOS: 111 | mRNA. /FEA = mRNA /GEN = KPNB2 | |
| (DNA) and 258 | /PROD = karyopherin (importin) beta 2 | |
| (amino acid) | /DB_XREF = gi: 4504906 /UG = Hs.168075 | |
| | karyopherin (importin) beta 2 | |
| | /FL = gb: U70322.1 gb: NM_002270.1 | |
| PTPRE: protein | Consensus includes gb: AA775177 /FEA = EST | 221840_at |
| tyrosine phosphatase, | /DB_XREF = gi: 2834511 | |
| receptor type, E | /DB_XREF = est: ac79a06.s1 | |
| isoform 1 precursor | /CLONE = IMAGE: 868786 /UG = Hs.31137 | |
| (LOC5791) | protein tyrosine phosphatase, receptor type, E | |
| SEQ ID NOS: 112 | /FL = gb: NM_006504.1 | |
| (DNA) and 259 | | |
| (amino acid) | | |
| TRB2: tribbles | gb: NM_021643.1 /DEF = Homo sapiens GS3955 | 202478_at |
| homolog 2 | protein (GS3955), mRNA. /FEA = mRNA | |
| (LOC28951) | /GEN = GS3955 /PROD = GS3955 protein | |
| SEQ ID NOS: 113 | /DB_XREF = gi: 11056053 /UG = Hs.155418 | |
| (DNA) and 260 | GS3955 protein /FL = gb: NM_021643.1 | |
| (amino acid) | gb: BC002637.1 gb: D87119.1 | |
| COL13A1: alpha 1 | gb: M33653.1 /DEF = Human (clones HT- | 211343_s_at |
| type XIII collagen | 125,133) alpha-2 type IV collagen (COL4A2) | |
| isoform 1 (LOC130) | mRNA, complete cds. /FEA = mRNA | |
| SEQ ID NOS: 114 | /GEN = COL4A2 /PROD = alpha-2 type IV | |
| (DNA) and 261 | collagen /DB_XREF = gi: 180828 | |
| (amino acid) | /UG = Hs.211933 collagen, type XIII, alpha 1 | |
| | /FL = gb: M33653.1 | |
| PALM2: paralemmin | gb: NM_007203.1 /DEF = Homo sapiens A | 202760_s_at |
| 2 (LOC114299) | kinase (PRKA) anchor protein 2 (AKAP2), | |
| SEQ ID NOS: 115 | mRNA. /FEA = mRNA /GEN = AKAP2 | |
| (DNA) and 262 | /PROD = A kinase (PRKA) anchor protein 2 | |
| (amino acid) | /DB_XREF = gi: 6005708 /UG = Hs.42322 A | |
| | kinase (PRKA) anchor protein 2 | |
| | /FL = gb: AB023137.1 gb: NM_007203.1 | |
| GJA1: connexin 43 | gb: NM_000165.2 /DEF = Homo sapiens gap | 201667_at |
| (LOC2697) | junction protein, alpha 1, 43 kD (connexin 43) | |
| SEQ ID NOS: 116 | (GJA1), mRNA. /FEA = mRNA /GEN = GJA1 | |
| (DNA) and 263 | /PROD = connexin 43 /DB_XREF = gi: 4755136 | |
| (amino acid) | /UG = Hs.74471 gap junction protein, alpha 1, | |
| | 43 kD (connexin 43) /FL = gb: M65188.1 | |
| | gb: NM_000165.2 | |
| FLJ10901: | gb: NM_018265.1 /DEF = Homo sapiens | 219010_at |
| hypothetical protein | hypothetical protein FLJ10901 (FLJ10901), | |
| FLJ10901 | mRNA. /FEA = mRNA /GEN = FLJ10901 | |
| (LOC55765) | /PROD = hypothetical protein FLJ10901 | |
| SEQ ID NOS: 117 | /DB_XREF = gi: 8922753 /UG = Hs.73239 | |
| (DNA) and 264 | hypothetical protein FLJ10901 | |
| (amino acid) | /FL = gb: NM_018265.1 | |
| EFEMP1: EGF- | Consensus includes gb: AI826799 /FEA = EST | 201842_s_at |
| containing fibulin- | /DB_XREF = gi: 5447470 | |
| like extracellular | /DB_XREF = est: wk56d07.x1 | |
| matrix protein 1 | /CLONE = IMAGE: 2419405 /UG = Hs.76224 | |
| isoform a precursor | EGF-containing fibulin-like extracellular matrix | |
| (LOC2202) | protein 1 /FL = gb: U03877.1 gb: NM_004105.2 | |
| SEQ ID NOS: 118 | | |
| (DNA) and 265 | | |
| (amino acid) | | |
| NRP1: neuropilin 1 | Consensus includes gb: BE620457 /FEA = EST | 212298_at |
| (LOC8829) | /DB_XREF = gi: 9891395 | |
| SEQ ID NOS: 119 | /DB_XREF = est: 601483690F1 | |
| (DNA) and 266 | /CLONE = IMAGE: 3886055 /UG = Hs.69285 | |
| (amino acid) | neuropilin 1 /FL = gb: AF018956.1 | |
| | gb: AF016050.1 gb: NM_003873.1 | |
| CLDN7: claudin 7 | gb: NM_001307.1 /DEF = Homo sapiens claudin | 202790_at |
| (LOC1366) | 7 (CLDN7), mRNA. /FEA = mRNA | |
| SEQ ID NOS: 120 | /GEN = CLDN7 /PROD = claudin 7 | |
| (DNA) and 267 | /DB_XREF = gi: 10835007 /UG = Hs.278562 | |
| (amino acid) | claudin 7 /FL = gb: NM_001307.1 | |
| | gb: BC001055.1 | |
| CED-6: PTB domain | gb: NM_016315.1 /DEF = Homo sapiens CED-6 | 204237_at |
| adaptor protein CED- | protein (CED-6), mRNA. /FEA = mRNA | |
| 6 (LOC51454) | /GEN = CED-6 /PROD = CED-6 protein | |
| SEQ ID NOS: 121 | /DB_XREF = gi: 7705317 /UG = Hs.107056 CED- | |
| (DNA) and 268 | 6 protein /FL = gb: AF200715.1 gb: AF191771.1 | |
| (amino acid) | gb: NM_016315.1 | |
| CSPG2: chondroitin | Consensus includes gb: BF590263 /FEA = EST | 204619_s_at |
| sulfate proteoglycan 2 | /DB_XREF = gi: 11682587 | |
| (versican) (LOC1462) | /DB_XREF = est: nab22b12.x1 | |
| SEQ ID NOS: 122 | /CLONE = IMAGE: 3266638 /UG = Hs.81800 | |
| (DNA) and 269 | chondroitin sulfate proteoglycan 2 (versican) | |
| (amino acid) | /FL = gb: NM_004385.1 | |
| KPNB2: karyopherin | gb: U72069.1 /DEF = Human karyopherin beta2 | 209226_s_at |
| beta 2 (LOC3842) | mRNA, complete cds. /FEA = mRNA | |
| SEQ ID NOS: 123 | /PROD = karyopherin beta2 | |
| (DNA) and 270 | /DB_XREF = gi: 1657775 /UG = Hs.168075 | |
| (amino acid) | karyopherin (importin) beta 2 | |
| | /FL = gb: U72069.1 gb: U72395.1 | |
| MLAT4: myxoid | gb: NM_018192.1 /DEF = Homo sapiens | 218717_s_at |
| liposarcoma | hypothetical protein FLJ10718 (FLJ10718), | |
| associated protein 4 | mRNA. /FEA = mRNA /GEN = FLJ10718 | |
| (LOC55214) | /PROD = hypothetical protein FLJ10718 | |
| SEQ ID NOS: 124 | /DB_XREF = gi: 8922618 /UG = Hs.42824 | |
| (DNA) and 271 | hypothetical protein FLJ10718 | |
| (amino acid) | /FL = gb: NM_018192.1 | |
| TPM1: tropomyosin 1 | gb: Z24727.1 /DEF = H. sapiens tropomyosin | 210986_s_at |
| (alpha) (LOC7168) | isoform mRNA, complete CDS. /FEA = mRNA | |
| SEQ ID NOS: 125 | /PROD = tropomyosin isoform | |
| (DNA) and 272 | /DB_XREF = gi: 854188 /UG = Hs.77899 | |
| (amino acid) | tropomyosin 1 (alpha) /FL = gb: Z24727.1 | |
| LY96: MD-2 protein | gb: NM_015364.1 /DEF = Homo sapiens MD-2 | 206584_at |
| (LOC23643) | protein (MD-2), mRNA. /FEA = mRNA | |
| SEQ ID NOS: 126 | /GEN = MD-2 /PROD = MD-2 protein | |
| (DNA) and 273 | /DB_XREF = gi: 7662503 /UG = Hs.69328 MD-2 | |
| (amino acid) | protein /FL = gb: AB018549.1 gb: NM_015364.1 | |
| | gb: AF168121.1 | |
| COL6A1: collagen, | Consensus includes gb: AI141603 /FEA = EST | 212091_s_at |
| type VI, alpha 1 | /DB_XREF = gi: 3649060 | |
| precursor (LOC1291) | /DB_XREF = est: qa90h10.x1 | |
| SEQ ID NOS: 127 | /CLONE = IMAGE: 1694083 /UG = Hs.108885 | |
| (DNA) and 274 | collagen, type VI, alpha 1 | |
| (amino acid) | | |
| CDC42EP3: Cdc42 | gb: AL136842.1 /DEF = Homo sapiens mRNA; | 209288_s_at |
| effector protein 3 | cDNA DKFZp434A0530 (from clone | |
| (LOC10602) | DKFZp434A0530); complete cds. | |
| SEQ ID NOS: 128 | /FEA = mRNA /GEN = DKFZp434A0530 | |
| (DNA) and 275 | /PROD = hypothetical protein | |
| (amino acid) | /DB_XREF = gi: 6807668 /UG = Hs.260024 | |
| | Cdc42 effector protein 3 /FL = gb: AF094521.1 | |
| | gb: AF104857.1 gb: NM_006449.1 | |
| | gb: AF164118.1 gb: AL136842.1 | |
| JTB: jumping | gb: NM_006694.1 /DEF = Homo sapiens | 200048_s_at |
| translocation | jumping translocation breakpoint (JTB), | |
| breakpoint | mRNA. /FEA = mRNA /GEN = JTB | |
| (LOC10899) | /PROD = jumping translocation breakpoint | |
| SEQ ID NOS: 129 | /DB_XREF = gi: 5729888 /UG = Hs.6396 jumping | |
| (DNA) and 276 | translocation breakpoint /FL = gb: BC000499.1 | |
| (amino acid) | gb: BC001363.1 gb: BC000996.2 | |
| | gb: BC001667.1 gb: AB016488.1 | |
| | gb: AF131797.1 gb: NM_006694.1 | |
| | gb: AF115850.2 | |
| CDH2: cadherin 2, | gb: M34064.1 /DEF = Human N-cadherin | 203440_at |
| type 1 preproprotein | mRNA, complete cds. /FEA = mRNA | |
| (LOC1000) | /GEN = NCAD /DB_XREF = gi: 416292 | |
| SEQ ID NOS: 130 | /UG = Hs.161 cadherin 2, type 1, N-cadherin | |
| (DNA) and 277 | (neuronal) /FL = gb: M34064.1 gb: NM_001792.1 | |
| (amino acid) | | |
| MYLK: myosin light | gb: NM_005965.1 /DEF = Homo sapiens myosin, | 202555_s_at |
| chain kinase isoform | light polypeptide kinase (MYLK), mRNA. | |
| 6 (LOC4638) | /FEA = mRNA /GEN = MYLK /PROD = myosin, | |
| SEQ ID NOS: 131 | light polypeptide kinase | |
| (DNA) and 278 | /DB_XREF = gi: 5174600 /UG = Hs.211582 | |
| (amino acid) | myosin, light polypeptide kinase | |
| | /FL = gb: AB037663.1 gb: NM_005965.1 | |
| | gb: AF069601.2 | |
| COL4A1: alpha 1 | Consensus includes gb: NM_001845.1 | 211981_at |
| type IV collagen | /DEF = Homo sapiens collagen, type IV, alpha 1 | |
| preproprotein | (COL4A1), mRNA. /FEA = CDS | |
| (LOC1282) | /GEN = COL4A1 /PROD = collagen, type IV, | |
| SEQ ID NOS: 132 | alpha 1 /DB_XREF = gi: 7656984 | |
| (DNA) and 279 | /UG = Hs.119129 collagen, type IV, alpha 1 | |
| (amino acid) | /FL = gb: NM_001845.1 | |
| PROS1: protein S | gb: NM_000313.1 /DEF = Homo sapiens protein | 207808_s_at |
| (alpha) (LOC5627) | S (alpha) (PROS1), mRNA. /FEA = mRNA | |
| SEQ ID NOS: 133 | /GEN = PROS1 /PROD = protein S (alpha) | |
| (DNA) and 280 | /DB_XREF = gi: 4506116 /UG = Hs.64016 protein | |
| (amino acid) | S (alpha) /FL = gb: M15036.1 gb: NM_000313.1 | |
| EFEMP1: EGF- | gb: NM_004105.2 /DEF = Homo sapiens EGF- | 201843_s_at |
| containing fibulin- | containing fibulin-like extracellular matrix | |
| like extracellular | protein 1 (EFEMP1), transcript variant 1, | |
| matrix protein 1 | mRNA. /FEA = mRNA /GEN = EFEMP1 | |
| isoform a precursor | /PROD = EGF-containing fibulin-like | |
| (LOC2202) | extracellular matrixprotein 1 precursor, isoform | |
| SEQ ID NOS: 134 | a precursor /DB_XREF = gi: 9665261 | |
| (DNA) and 281 | /UG = Hs.76224 EGF-containing fibulin-like | |
| (amino acid) | extracellular matrix protein 1 /FL = gb: U03877.1 | |
| | gb: NM_004105.2 | |
| CCL2: small | Consensus includes gb: S69738.1 /DEF = MCP- | 216598_s_at |
| inducible cytokine A2 | 1 = monocyte chemotactic protein human, aortic | |
| precursor (LOC6347) | endothelial cells, mRNA, 661 nt. /FEA = mRNA | |
| SEQ ID NOS: 135 | /GEN = MCP-1 /PROD = MCP-1 | |
| (DNA) and 282 | /DB_XREF = gi: 545464 /UG = Hs.303649 small | |
| (amino acid) | inducible cytokine A2 (monocyte chemotactic | |
| | protein 1, homologous to mouse Sig-je) | |
| DFNA5: deafness, | gb: NM_004403.1 /DEF = Homo sapiens | 203695_s_at |
| autosomal dominant 5 | deafness, autosomal dominant 5 (DFNA5), | |
| protein (LOC1687) | mRNA. /FEA = mRNA /GEN = DFNA5 | |
| SEQ ID NOS: 136 | /PROD = deafness, autosomal dominant 5 | |
| (DNA) and 283 | protein /DB_XREF = gi: 4758153 /UG = Hs.13530 | |
| (amino acid) | deafness, autosomal dominant 5 | |
| | /FL = gb: AF073308.1 gb: NM_004403.1 | |
| | gb: AF007790.2 | |
| TPM1: tropomyosin 1 | gb: M19267.1 /DEF = Human tropomyosin | 210987_x_at |
| (alpha) (LOC7168) | mRNA, complete cds. /FEA = mRNA | |
| SEQ ID NOS: 137 | /DB_XREF = gi: 339943 /UG = Hs.77899 | |
| (DNA) and 284 | tropomyosin 1 (alpha) /FL = gb: M19267.1 | |
| (amino acid) | | |
| DDAH1: | Consensus includes gb: AL078459 | 209094_at |
| dimethylarginine | /DEF = Human DNA sequence from clone RP4- | |
| dimethylaminohydrolase | 621F18 on chromosome 1p11.4-21.3. Contains | |
| 1 (LOC23576) | the 3 end of the gene for ng, ng | |
| SEQ ID NOS: 138 | dimethylarginine dimethylaminohydrolase (EC | |
| (DNA) and 285 | 3.5.3.18), ESTs, STSs and GSSs /FEA = mRNA | |
| (amino acid) | /DB_XREF = gi: 5791502 /UG = Hs.303180 | |
| | dimethylarginine dimethylaminohydrolase 1 | |
| | /FL = gb: AB001915.1 gb: NM_012137.1 | |
| PMAIP1: phorbol-12- | Consensus includes gb: AI857639 /FEA = EST | 204285_s_at |
| myristate-13-acetate- | /DB_XREF = gi: 5511255 | |
| induced protein 1 | /DB_XREF = est: wk95g09.x1 | |
| (LOC5366) | /CLONE = IMAGE: 2423200 /UG = Hs.96 | |
| SEQ ID NOS: 139 | phorbol-12-myristate-13-acetate-induced | |
| (DNA) and 286 | protein 1 /FL = gb: NM_021127.1 | |
| (amino acid) | | |
| ACOX2: acyl- | gb: NM_003500.1 /DEF = Homo sapiens acyl- | 205364_at |
| Coenzyme A oxidase | Coenzyme A oxidase 2, branched chain | |
| 2, branched chain | (ACOX2), mRNA. /FEA = mRNA | |
| (LOC8309) | /GEN = ACOX2 /PROD = acyl-Coenzyme A | |
| SEQ ID NOS: 140 | oxidase 2, branched chain | |
| (DNA) and 287 | /DB_XREF = gi: 4501868 /UG = Hs.9795 acyl- | |
| (amino acid) | Coenzyme A oxidase 2, branched chain | |
| | /FL = gb: NM_003500.1 | |
| GDI1: GDP | gb: NM_001493.1 /DEF = Homo sapiens GDP | 201864_at |
| dissociation inhibitor | dissociation inhibitor 1 (GDI1), mRNA. | |
| 1 (LOC2664) | /FEA = mRNA /GEN = GDI1 /PROD = GDP | |
| SEQ ID NOS: 141 | dissociation inhibitor 1 /DB_XREF = gi: 4503970 | |
| (DNA) and 288 | /UG = Hs.74576 GDP dissociation inhibitor 1 | |
| (amino acid) | /FL = gb: BC000317.1 gb: NM_001493.1 | |
| | gb: D45021.1 | |
| DPYSL3: | gb: NM_001387.1 /DEF = Homo sapiens | 201431_s_at |
| dihydropyrimidinase- | dihydropyrimidinase-like 3 (DPYSL3), mRNA. | |
| like 3 (LOC1809) | /FEA = mRNA /GEN = DPYSL3 | |
| SEQ ID NOS: 142 | /PROD = dihydropyrimidinase-like 3 | |
| (DNA) and 289 | /DB_XREF = gi: 4503378 /UG = Hs.74566 | |
| (amino acid) | dihydropyrimidinase-like 3 /FL = gb: D78014.1 | |
| | gb: NM_001387.1 | |
| APOC1: | Consensus includes gb: W79394 /FEA = EST | 213553_x_at |
| apolipoprotein C-I | /DB_XREF = gi: 1390665 | |
| precursor (LOC341) | /DB_XREF = est: zd80c07.s1 | |
| SEQ ID NOS: 143 | /CLONE = IMAGE: 346956 /UG = Hs.268571 | |
| (DNA) and 290 | apolipoprotein C-I | |
| (amino acid) | | |
| TTC3: | gb: NM_003316.1 /DEF = Homo sapiens | 208073_x_at |
| tetratricopeptide | tetratricopeptide repeat domain 3 (TTC3), | |
| repeat domain 3 | mRNA. /FEA = mRNA /GEN = TTC3 | |
| (LOC7267) | /PROD = tetratricopeptide repeat domain 3 | |
| SEQ ID NOS: 144 | /DB_XREF = gi: 10835036 /UG = Hs.118174 | |
| (DNA) and 291 | tetratricopeptide repeat domain 3 | |
| (amino acid) | /FL = gb: NM_003316.1 gb: D84295.1 | |
| SNX6: sorting nexin | gb: NM_021249.1 /DEF = Homo sapiens sorting | 217789_at |
| 6 isoform a | nexin 6 (SNX6), mRNA. /FEA = mRNA | |
| (LOC58533) | /GEN = SNX6 /PROD = sorting nexin 6 | |
| SEQ ID NOS: 145 | /DB_XREF = gi: 13027619 /UG = Hs.284291 | |
| (DNA) and 292 | sorting nexin 6 /FL = gb: BC001798.1 | |
| (amino acid) | gb: NM_021249.1 gb: AF121856.1 | |
| CKAP4: | Consensus includes gb: AW029619 /FEA = EST | 200998_s_at |
| transmembrane | /DB_XREF = gi: 5888375 | |
| protein (63 kD), | /DB_XREF = est: wx14e05.x1 | |
| endoplasmic | /CLONE = IMAGE: 2543648 /UG = Hs.74368 | |
| reticulum/Golgi | transmembrane protein (63 kD), endoplasmic | |
| interm (LOC10970) | reticulumGolgi intermediate compartment | |
| SEQ ID NOS: 146 | /FL = gb: NM_006825.1 | |
| (DNA) and 293 | | |
| (amino acid) | | |
| TUBB: tubulin, beta | gb: NM_001069.1 /DEF = Homo sapiens tubulin, | 204141_at |
| polypeptide | beta polypeptide (TUBB), mRNA. | |
| (LOC7280) | /FEA = mRNA /GEN = TUBB /PROD = tubulin, | |
| SEQ ID NOS: 147 | beta polypeptide /DB_XREF = gi: 4507728 | |
| (DNA) and 294 | /UG = Hs.179661 tubulin, beta polypeptide | |
| (amino acid) | /FL = gb: BC001194.1 gb: NM_001069.1 | |

The biomarkers provided in Table 1, which include the nucleotide sequences of SEQ ID NOS:1-147 and the amino acid sequences of SEQ ID NOS:148-294, referred to herein as a total of 147 biomarkers with reference to the Unigene Title, includes 40 cases where multiple probe sets measure the intensity of a single biomarker (at most, three probe sets for one biomarker). In these cases, the redundant probe sets reference the same full-length cDNA and protein sequences. Table 2 provides a correlation between the NCBI locus IDs and the probe set IDs.

TABLE 2
Correlation between NCBI Locus IDs and Probe Set IDs

| NCBI | Number of | |
|---|---|---|
| Locus ID | Probe sets | Probe set IDs |
| 182 | 3 | 209099_x_at, 209098_s_at, 216268_s_at |
| 1462 | 3 | 204620_s_at, 221731_x_at, 204619_s_at |
| 2316 | 3 | 214752_x_at, 213746_s_at, 200859_x_at |
| 3842 | 3 | 221829_s_at, 207657_x_at, 209226_s_at |
| 9060 | 3 | 203060_s_at, 203059_s_at, 203058_s_at |
| 10899 | 3 | 210434_x_at, 210927_x_at, 200048_s_at |
| 214 | 2 | 201952_at, 201951_at |
| 1291 | 2 | 213428_s_at, 212091_s_at |
| 1508 | 2 | 200839_s_at, 200838_at |
| 1809 | 2 | 201430_s_at, 201431_s_at |
| 2202 | 2 | 201842_s_at, 201843_s_at |
| 2810 | 2 | 33322_i_at, 33323_r_at |
| 3428 | 2 | 206332_s_at, 208966_x_at |
| 3491 | 2 | 201289_at, 210764_s_at |
| 7168 | 2 | 210986_s_at, 210987_x_at |
| 8781 | 2 | 212509_s_at, 205048_s_at |
| 10628 | 2 | 201008_s_at, 201010_s_at |
| 130 | 1 | 211343_s_at |
| 309 | 1 | 200982_s_at |
| 341 | 1 | 213553_x_at |
| 754 | 1 | 200677_at |
| 800 | 1 | 212077_at |
| 999 | 1 | 201131_s_at |
| 1000 | 1 | 203440_at |
| 1075 | 1 | 201487_at |
| 1282 | 1 | 211981_at |
| 1292 | 1 | 209156_s_at |
| 1293 | 1 | 201438_at |
| 1366 | 1 | 202790_at |
| 1490 | 1 | 209101_at |
| 1687 | 1 | 203695_s_at |
| 1808 | 1 | 200762_at |
| 1836 | 1 | 205097_at |
| 2014 | 1 | 203729_at |
| 2115 | 1 | 221911_at |
| 2131 | 1 | 201995_at |
| 2150 | 1 | 213506_at |
| 2273 | 1 | 201540_at |
| 2591 | 1 | 203397_s_at |
| 2664 | 1 | 201864_at |
| 2697 | 1 | 201667_at |
| 2791 | 1 | 204115_at |
| 2887 | 1 | 209409_at |
| 3091 | 1 | 200989_at |
| 3371 | 1 | 201645_at |
| 3383 | 1 | 202637_s_at |
| 3576 | 1 | 211506_s_at |
| 3685 | 1 | 202351_at |
| 3728 | 1 | 201015_s_at |
| 3855 | 1 | 209016_s_at |
| 3856 | 1 | 209008_x_at |
| 3875 | 1 | 201596_x_at |
| 3880 | 1 | 201650_at |
| 3916 | 1 | 201553_s_at |
| 4017 | 1 | 202998_s_at |
| 4131 | 1 | 212233_at |
| 4192 | 1 | 209035_at |
| 4638 | 1 | 202555_s_at |
| 5066 | 1 | 202336_s_at |
| 5270 | 1 | 212190_at |
| 5366 | 1 | 204285_s_at |
| 5376 | 1 | 210139_s_at |
| 5441 | 1 | 211730_s_at |
| 5621 | 1 | 201300_s_at |
| 5627 | 1 | 207808_s_at |
| 5791 | 1 | 221840_at |
| 5834 | 1 | 201481_s_at |
| 6137 | 1 | 212191_x_at |
| 6159 | 1 | 213969_x_at |
| 6280 | 1 | 203535_at |
| 6303 | 1 | 210592_s_at |
| 6347 | 1 | 216598_s_at |
| 6382 | 1 | 201287_s_at |
| 6535 | 1 | 202219_at |
| 6678 | 1 | 200665_s_at |
| 6692 | 1 | 202826_at |
| 6748 | 1 | 201004_at |
| 6768 | 1 | 202005_at |
| 6772 | 1 | 200887_s_at |
| 7045 | 1 | 201506_at |
| 7267 | 1 | 208073_x_at |
| 7280 | 1 | 204141_at |
| 7296 | 1 | 201266_at |
| 7298 | 1 | 202589_at |
| 7345 | 1 | 201387_s_at |
| 7358 | 1 | 203343_at |
| 7846 | 1 | 209118_s_at |
| 8309 | 1 | 205364_at |
| 8829 | 1 | 212298_at |
| 9263 | 1 | 202693_s_at |
| 9540 | 1 | 210609_s_at |
| 10135 | 1 | 217738_at |
| 10602 | 1 | 209288_s_at |
| 10653 | 1 | 210715_s_at |
| 10962 | 1 | 211071_s_at |
| 10970 | 1 | 200998_s_at |
| 11098 | 1 | 202458_at |
| 11199 | 1 | 210143_at |
| 22943 | 1 | 204602_at |
| 23231 | 1 | 212314_at |
| 23362 | 1 | 203354_s_at |
| 23576 | 1 | 209094_at |
| 23643 | 1 | 206584_at |
| 25932 | 1 | 201560_at |
| 26064 | 1 | 202052_s_at |
| 26751 | 1 | 204019_s_at |
| 27122 | 1 | 214247_s_at |
| 28951 | 1 | 202478_at |
| 29984 | 1 | 209885_at |
| 51065 | 1 | 218007_s_at |
| 51454 | 1 | 204237_at |
| 51809 | 1 | 218313_s_at |
| 54407 | 1 | 218041_x_at |
| 55214 | 1 | 218717_s_at |
| 55765 | 1 | 219010_at |
| 56034 | 1 | 218718_at |
| 57111 | 1 | 218186_at |
| 57402 | 1 | 218677_at |
| 58533 | 1 | 217789_at |
| 64759 | 1 | 217853_at |
| 84617 | 1 | 209191_at |
| 113146 | 1 | 212992_at |
| 114299 | 1 | 202760_s_at |
| 347902 | 1 | 222108_at |

The biomarkers have expression levels in the cells that may be dependent on the activity of the EGFR signal transduction pathway, and that are also highly correlated with EGFR modulator sensitivity exhibited by the cells. Biomarkers serve as useful molecular tools for predicting a response to EGFR modulators, preferably biological molecules, small molecules, and the like that affect EGFR kinase activity via direct or indirect inhibition or antagonism of EGFR kinase function or activity.

EGFR Modulators

As used herein, the term “EGFR modulator” is intended to mean a compound or drug that is a biological molecule or a small molecule that directly or indirectly modulates EGFR activity or the EGFR signal transduction pathway. Thus, compounds or drugs as used herein is intended to include both small molecules and biological molecules. Direct or indirect modulation includes activation or inhibition of EGFR activity or the EGFR signal transduction pathway. In one aspect, inhibition refers to inhibition of the binding of EGFR to an EGFR ligand such as, for example, EGF. In another aspect, inhibition refers to inhibition of the kinase activity of EGFR.

EGFR modulators include, for example, EGFR-specific ligands, small molecule EGFR inhibitors, and EGFR monoclonal antibodies. In one aspect, the EGFR modulator inhibits EGFR activity and/or inhibits the EGFR signal transduction pathway. In another aspect, the EGFR modulator is an EGFR monoclonal antibody that inhibits EGFR activity and/or inhibits the EGFR signal transduction pathway.

EGFR modulators include biological molecules or small molecules. Biological molecules include all lipids and polymers of monosaccharides, amino acids, and nucleotides having a molecular weight greater than 450. Thus, biological molecules include, for example, oligosaccharides and polysaccharides; oligopeptides, polypeptides, peptides, and proteins; and oligonucleotides and polynucleotides. Oligonucleotides and polynucleotides include, for example, DNA and RNA.

Biological molecules further include derivatives of any of the molecules described above. For example, derivatives of biological molecules include lipid and glycosylation derivatives of oligopeptides, polypeptides, peptides, and proteins.

Derivatives of biological molecules further include lipid derivatives of oligosaccharides and polysaccharides, e.g., lipopolysaccharides. Most typically, biological molecules are antibodies, or functional equivalents of antibodies. Functional equivalents of antibodies have binding characteristics comparable to those of antibodies, and inhibit the growth of cells that express EGFR. Such functional equivalents include, for example, chimerized, humanized, and single chain antibodies as well as fragments thereof.

Functional equivalents of antibodies also include polypeptides with amino acid sequences substantially the same as the amino acid sequence of the variable or hypervariable regions of the antibodies. An amino acid sequence that is substantially the same as another sequence, but that differs from the other sequence by means of one or more substitutions, additions, and/or deletions, is considered to be an equivalent sequence. Preferably, less than 50%, more preferably less than 25%, and still more preferably less than 10%, of the number of amino acid residues in a sequence are substituted for, added to, or deleted from the protein.

The functional equivalent of an antibody is preferably a chimerized or humanized antibody. A chimerized antibody comprises the variable region of a non-human antibody and the constant region of a human antibody. A humanized antibody comprises the hypervariable region (CDRs) of a non-human antibody. The variable region other than the hypervariable region, e.g., the framework variable region, and the constant region of a humanized antibody are those of a human antibody.

Suitable variable and hypervariable regions of non-human antibodies may be derived from antibodies produced by any non-human mammal in which monoclonal antibodies are made. Suitable examples of mammals other than humans include, for example, rabbits, rats, mice, horses, goats, or primates.

Functional equivalents further include fragments of antibodies that have binding characteristics that are the same as, or are comparable to, those of the whole antibody. Suitable fragments of the antibody include any fragment that comprises a sufficient portion of the hypervariable (i.e., complementarity determining) region to bind specifically, and with sufficient affinity, to EGFR tyrosine kinase to inhibit growth of cells that express such receptors.

Such fragments may, for example, contain one or both Fab fragments or the F(ab′)₂ fragment. Preferably, the antibody fragments contain all six complementarity determining regions of the whole antibody, although functional fragments containing fewer than all of such regions, such as three, four, or five CDRs, are also included.

In one aspect, the fragments are single chain antibodies, or Fv fragments. Single chain antibodies are polypeptides that comprise at least the variable region of the heavy chain of the antibody linked to the variable region of the light chain, with or without an interconnecting linker. Thus, Fv fragment comprises the entire antibody combining site. These chains may be produced in bacteria or in eukaryotic cells.

The antibodies and functional equivalents may be members of any class of immunoglobulins, such as IgG, IgM, IgA, IgD, or IgE, and the subclasses thereof.

In one aspect, the antibodies are members of the IgG1 subclass. The functional equivalents may also be equivalents of combinations of any of the above classes and subclasses.

In one aspect, EGFR antibodies can be selected from chimerized, humanized, fully human, and single chain antibodies derived from the murine antibody 225 described in U.S. Pat. No. 4,943,533 to Mendelsohn et al.

In another aspect, the EGFR antibody can be selected from the antibodies described in U.S. Pat. No. 6,235,883 to Jakobovits et al., U.S. Pat. No. 5,558,864 to Bendi et al., and U.S. Pat. No. 5,891,996 to Mateo de Acosta del Rio et al.

In addition to the biological molecules discussed above, the EGFR modulators useful in the invention may also be small molecules. Any molecule that is not a biological molecule is considered herein to be a small molecule. Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds. In one embodiment, the EGFR modulator is a small molecule that inhibits the growth of tumor cells that express EGFR. In another embodiment, the EGFR modulator is a small molecule that inhibits the growth of refractory tumor cells that express EGFR.

Numerous small molecules have been described as being useful to inhibit EGFR. For example, U.S. Pat. No. 5,656,655 to Spada et al. discloses styryl substituted heteroaryl compounds that inhibit EGFR. The heteroaryl group is a monocyclic ring with one or two heteroatoms, or a bicyclic ring with 1 to about 4 heteroatoms, the compound being optionally substituted or polysubstituted.

U.S. Pat. No. 5,646,153 to Spada et al. discloses bis mono and/or bicyclic aryl heteroaryl, carbocyclic, and heterocarbocyclic compounds that inhibit EGFR.

U.S. Pat. No. 5,679,683 to Bridges et al. discloses tricyclic pyrimidine compounds that inhibit the EGFR. The compounds are fused heterocyclic pyrimidine derivatives described at column 3, line 35 to column 5, line 6.

U.S. Pat. No. 5,616,582 to Barker discloses quinazoline derivatives that have receptor tyrosine kinase inhibitory activity.

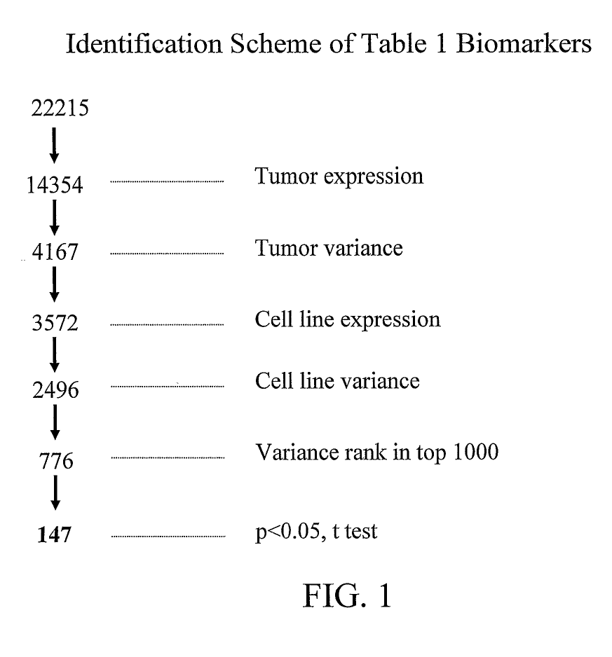

Fry et al., Science 265, 1093-1095 (1994) in FIG. 1 discloses a compound having a structure that inhibits EGFR.

Osherov et al. disclose tyrphostins that inhibit EGFR/HER1 and HER 2, particularly those in Tables I, II, III, and IV.

U.S. Pat. No. 5,196,446 to Levitzki et al. discloses heteroarylethenediyl or heteroarylethendeiylaryl compounds that inhibit EGFR, particularly from column 2, line 42 to column 3, line 40.

Panek et al., Journal of Pharmacology and Experimental Therapeutics 283, 1433-1444 (1997) discloses a compound identified as PD166285 that inhibits the EGFR, PDGFR, and FGFR families of receptors. PD166285 is identified as 6-(2,6-dichlorophenyl)-2-(4-(2-diethylaminoethyoxy)phenylamino)-8-methyl-8H-pyrido(2,3-d)pyrimidin-7-one having the structure shown in FIG. 1 on page 1436.

Biomarkers and Biomarker Sets

The invention includes individual biomarkers and biomarker sets having both diagnostic and prognostic value in disease areas in which signaling through EGFR or the EGFR pathway is of importance, e.g., in cancers or tumors, in immunological disorders, conditions or dysfunctions, or in disease states in which cell signaling and/or cellular proliferation controls are abnormal or aberrant. The biomarker sets comprise a plurality of biomarkers such as, for example, a plurality of the biomarkers provided in Table 1, that highly correlate with resistance or sensitivity to one or more EGFR modulators.

The biomarker sets of the invention enable one to predict or reasonably foretell the likely effect of one or more EGFR modulators in different biological systems or for cellular responses. The biomarker sets can be used in in vitro assays of EGFR modulator response by test cells to predict in vivo outcome. In accordance with the invention, the various biomarker sets described herein, or the combination of these biomarker sets with other biomarkers or markers, can be used, for example, to predict how patients with cancer might respond to therapeutic intervention with one or more EGFR modulators.

A biomarker set of cellular gene expression patterns correlating with sensitivity or resistance of cells following exposure of the cells to one or more EGFR modulators provides a useful tool for screening one or more tumor samples before treatment with the EGFR modulator. The screening allows a prediction of cells of a tumor sample exposed to one or more EGFR modulators, based on the expression results of the biomarker set, as to whether or not the tumor, and hence a patient harboring the tumor, will or will not respond to treatment with the EGFR modulator.

The biomarker or biomarker set can also be used as described herein for monitoring the progress of disease treatment or therapy in those patients undergoing treatment for a disease involving an EGFR modulator.

The biomarkers also serve as targets for the development of therapies for disease treatment. Such targets may be particularly applicable to treatment of lung disease, such as non-small cell lung cancers or tumors. Indeed, because these biomarkers are differentially expressed in sensitive and resistant cells, their expression patterns are correlated with relative intrinsic sensitivity of cells to treatment with EGFR modulators. Accordingly, the biomarkers highly expressed in resistant cells may serve as targets for the development of new therapies for the tumors which are resistant to EGFR modulators, particularly EGFR inhibitors.

The level of biomarker protein and/or mRNA can be determined using methods well known to those skilled in the art. For example, quantification of protein can be carried out using methods such as ELISA, 2-dimensional SDS PAGE, Western blot, immunopreciptation, immunohistochemistry, fluorescence activated cell sorting (FACS), or flow cytometry. Quantification of mRNA can be carried out using methods such as PCR, array hybridization, Northern blot, in-situ hybridization, dot-blot, Taqman, or RNAse protection assay.

Microarrays

The invention also includes specialized microarrays, e.g., oligonucleotide microarrays or cDNA microarrays, comprising one or more biomarkers, showing expression profiles that correlate with either sensitivity or resistance to one or more EGFR modulators. Such microarrays can be employed in in vitro assays for assessing the expression level of the biomarkers in the test cells from tumor biopsies, and determining whether these test cells are likely to be resistant or sensitive to EGFR modulators. For example, a specialized microarray can be prepared using all the biomarkers, or subsets thereof, as described herein and shown in Table 1. Cells from a tissue or organ biopsy can be isolated and exposed to one or more of the EGFR modulators. Following application of nucleic acids isolated from both untreated and treated cells to one or more of the specialized microarrays, the pattern of gene expression of the tested cells can be determined and compared with that of the biomarker pattern from the control panel of cells used to create the biomarker set on the microarray. Based upon the gene expression pattern results from the cells that underwent testing, it can be determined if the cells show a resistant or a sensitive profile of gene expression. Whether or not the tested cells from a tissue or organ biopsy will respond to one or more of the EGFR modulators and the course of treatment or therapy can then be determined or evaluated based on the information gleaned from the results of the specialized microarray analysis.

Antibodies

The invention also includes antibodies, including polyclonal or monoclonal, directed against one or more of the polypeptide biomarkers. Such antibodies can be used in a variety of ways, for example, to purify, detect, and target the biomarkers of the invention, including both in vitro and in vivo diagnostic, detection, screening, and/or therapeutic methods.

Kits

The invention also includes kits for determining or predicting whether a patient would be susceptible or resistant to a treatment that comprises one or more EGFR modulators. The patient may have a cancer or tumor such as, for example, a non-small cell lung cancer or tumor. Such kits would be useful in a clinical setting for use in testing a patient's biopsied tumor or other cancer samples, for example, to determine or predict if the patient's tumor or cancer will be resistant or sensitive to a given treatment or therapy with an EGFR modulator. The kit comprises a suitable container that comprises: one or more microarrays, e.g., oligonucleotide microarrays or cDNA microarrays, that comprise those biomarkers that correlate with resistance and sensitivity to EGFR modulators, particularly EGFR inhibitors; one or more EGFR modulators for use in testing cells from patient tissue specimens or patient samples; and instructions for use. In addition, kits contemplated by the invention can further include, for example, reagents or materials for monitoring the expression of biomarkers of the invention at the level of mRNA or protein, using other techniques and systems practiced in the art such as, for example, RT-PCR assays, which employ primers designed on the basis of one or more of the biomarkers described herein, immunoassays, such as enzyme linked immunosorbent assays (ELISAs), immunoblotting, e.g., Western blots, or in situ hybridization, and the like, as further described herein.

Application of Biomarkers and Biomarker Sets

The biomarkers and biomarker sets may be used in different applications. Biomarker sets can be built from any combination of biomarkers listed in Table 1 to make predictions about the likely effect of any EGFR modulator in different biological systems. The various biomarkers and biomarkers sets described herein can be used, for example, as diagnostic or prognostic indicators in disease management, to predict how patients with cancer might respond to therapeutic intervention with compounds that modulate the EGFR, and to predict how patients might respond to therapeutic intervention that modulates signaling through the entire EGFR regulatory pathway.

The biomarkers have both diagnostic and prognostic value in diseases areas in which signaling through EGFR or the EGFR pathway is of importance, e.g., in immunology, or in cancers or tumors in which cell signaling and/or proliferation controls have gone awry.

In accordance with the invention, cells from a patient tissue sample, e.g., a tumor or cancer biopsy, can be assayed to determine the expression pattern of one or more biomarkers prior to treatment with one or more EGFR modulators. In one aspect, the tumor or cancer is NSCLC. Success or failure of a treatment can be determined based on the biomarker expression pattern of the cells from the test tissue (test cells), e.g., tumor or cancer biopsy, as being relatively similar or different from the expression pattern of a control set of the one or more biomarkers. Thus, if the test cells show a biomarker expression profile which corresponds to that of the biomarkers in the control panel of cells which are sensitive to the EGFR modulator, it is highly likely or predicted that the individual's cancer or tumor will respond favorably to treatment with the EGFR modulator. By contrast, if the test cells show a biomarker expression pattern corresponding to that of the biomarkers of the control panel of cells which are resistant to the EGFR modulator, it is highly likely or predicted that the individual's cancer or tumor will not respond to treatment with the EGFR modulator.

The invention also provides a method of monitoring the treatment of a patient having a disease treatable by one or more EGFR modulators. The isolated test cells from the patient's tissue sample, e.g., a tumor biopsy or tumor sample, can be assayed to determine the expression pattern of one or more biomarkers before and after exposure to an EGFR modulator wherein, preferably, the EGFR modulator is an EGFR inhibitor. The resulting biomarker expression profile of the test cells before and after treatment is compared with that of one or more biomarkers as described and shown herein to be highly expressed in the control panel of cells that are either resistant or sensitive to an EGFR modulator. Thus, if a patient's response is sensitive to treatment by an EGFR modulator, based on correlation of the expression profile of the one or biomarkers, the patient's treatment prognosis can be qualified as favorable and treatment can continue. Also, if, after treatment with an EGFR modulator, the test cells don't show a change in the biomarker expression profile corresponding to the control panel of cells that are sensitive to the EGFR modulator, it can serve as an indicator that the current treatment should be modified, changed, or even discontinued. This monitoring process can indicate success or failure of a patient's treatment with an EGFR modulator and such monitoring processes can be repeated as necessary or desired.

The biomarkers of the invention can be used to predict an outcome prior to having any knowledge about a biological system. Essentially, a biomarker can be considered to be a statistical tool. Biomarkers are useful primarily in predicting the phenotype that is used to classify the biological system.

Although the complete function of all of the biomarkers are not currently known, some of the biomarkers are likely to be directly or indirectly involved in the EGFR signaling pathway. In addition, some of the biomarkers may function in metabolic or other resistance pathways specific to the EGFR modulators tested. Notwithstanding, knowledge about the function of the biomarkers is not a requisite for determining the accuracy of a biomarker according to the practice of the invention.

### EXAMPLES

### Example 1

### Identification of Biomarkers

The biomarkers of Table 1 were identified using three particular approaches. The transcriptional profiling data from primary tumors and cell lines was examined to identify genes with expression that is highly variable across the tumors and cell lines. In addition, attempts were made to determine the IC₅₀ on a panel of cell lines in order to identify genes whose expression profiles correlate with sensitive/resistant classification based on IC₅₀ values. Furthermore, cell lines and xenograft models were treated with the chimeric EGFR antibody cetuximab (marketed as Erbitux®) and the small molecule EGFR inhibitor gefitinib to identify genes that are modulated by EGFR inhibitors.

NSCLC Tumors and Patients

RNAs from twenty-nine NSCLC adenocarcinoma tumors were obtained (Ardais Corporation, Somerville, Mass.). Adenocarcinomas are the most common sub-type of NSCLC. The median age of the patients was 65 years (range: 43-80 years). The tumors belonged to all size ranges T1-T4 and all stages ranging from Stage IA to Stage IV according to the AJCC classification.

Determination of Relative Drug Sensitivity in NSCLC Cell Lines:

The NSCLC cell lines were grown using standard cell culture conditions: DMEM supplemented to contain 10% fetal bovine serum, 100 IU/ml penicillin, 100 mg/ml streptomycin and 2 mM L-glutamine (all from Invitrogen Life Technologies, Carlsbad, Calif.). Fourteen non-small cell lung cancer cell lines were examined for their sensitivity to EGFR inhibitor monoclonal antibody cetuximab. Cytotoxicity was assessed in cells by BrdU Cell Proliferation calorimetric ELISA (Roche Applied Science, Indianapolis, Ind.). This is a calorimetric immunoassay for the quantification of cell proliferation based on the measurement of BrdU incorporation during DNA synthesis. To carry out the assays, the NSCLC cells were plated at 2500-5000 cells/well in 96 well microtiter plates and 24 hours later diluted monoclonal antibody drug was added. The concentrations for the EGFR inhibitor cetuximab used in the cytotoxicity assays was 5 μg/ml, 4 μg/ml, 2 μg/ml, 1 μg/ml and 0.5 μg/ml. The cells were incubated at 37° C. for 48 hours at which time the BrdU labeling reagent was added. After two hours the labeling medium was removed and cells were fixed and the DNA was denatured using a FixDenat solution. The anti-BrdU antibody conjugated with peroxidase was added and immune complexes were detected by the subsequent substrate reaction. The reaction product was quantified by measuring the absorbance of the samples in an ELISA reader at 450 μm. The greater the absorbency, the greater the number of live cells. Only two of the fourteen cell lines tested had an IC₅₀ between 4 and 5 μg/ml. The IC₅₀ is the drug concentration required to inhibit cell proliferation to 50% of that of untreated cells. Three to six independent BrdU assays were performed for each cell line.

Resistance/Sensitivity Classification:

FIG. 1 shows the mRNA level of the epidermal growth factor receptor gene as determined by expression profiling of fourteen NSCLC cell lines that were tested in the BrdU assays described above. Cell lines are shown in order of increasing sensitivity to cetuximab. As shown in FIG. 1, there is no correlation between EGFR level and sensitivity to cetuximab. Of the fourteen NSCLC cell lines tested, ChagoK1 and L2987 were the only two cell lines that consistently showed ≧50% inhibition of cell proliferation at the IC₅₀ concentration of cetuximab. Cell lines SW900, Calu6, SK-MES1, H838 and H661 showed significantly lower than 50% inhibition of cell proliferation at the doses of cetuximab that were tested. The remaining cell lines LX1, H522, H441, H226, A549, SK-LU1 and H2347 showed no inhibition of cell proliferation at the doses of cetuximab that were tested. For the analysis, cell lines ChagoK1 and L2987 were defined as sensitive and the remaining twelve cell lines were defined as resistant.

Gene Expression Profiling:

RNA for the NSCLC adenocarcinomas was purchased from a commercial vendor as described above. For the NSCLC cell lines, RNA was isolated from 50-70% confluent cells using the RNeasy kits (Qiagen, Valencia, Calif.). The quality of RNA was checked by measuring the 28S:18: ribosomal RNA ratio using an Agilent 2100 Bioanalyzer (Agilent Technologies, Rockville, Md.). Concentration of total RNA was determined spectrophotometrically. 5 or 10 ug of total RNA was used to prepare biotinylated probes according to the Affymetrix Genechip Expression Analysis Technical Manual. Targets were hybridized to human HG-U133A gene chips according to the manufacturer's instructions. Data were preprocessed using the MAS 5.0 software (Affymetrix, Santa Clara, Calif.). The trimmed mean intensity for each chip was scaled to 1,500 to account for minor differences in global chip intensity so that the overall expression level for each sample is comparable.

Data Analysis

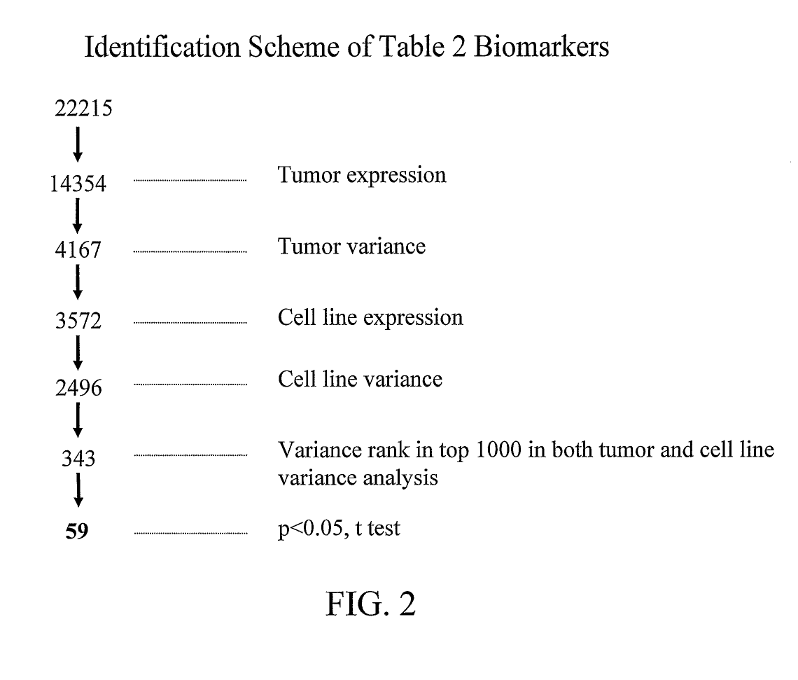

All 22,215 probes (gene sequences) present on the U133A chip were considered as potential predictive biomarkers. To restrict the analysis to gene sequences expressed in at least two of the twenty nine NSCLC tumors, gene sequences with Affymetrix MAS5.0 p>0.04 in at least two tumors or cell lines were removed leaving 14,354 and 13,909 gene sequences, respectively (FIG. 2).

Next, to identify genes with variable expression in lung tumors (and therefore more likely to be able to correlate with variability in response to treatment), a variance metric (the Weighted spread (90-10) metric) (WSpread (90-10) metric) was used to calculate the variance of probe sets in the tumor and cell line expression profiling data.

$$Weighted spread = \frac{I 90 th percentile - I 10 th percentile}{\sqrt{Imedian}}$$

I=Signal intensity from expression profiling data

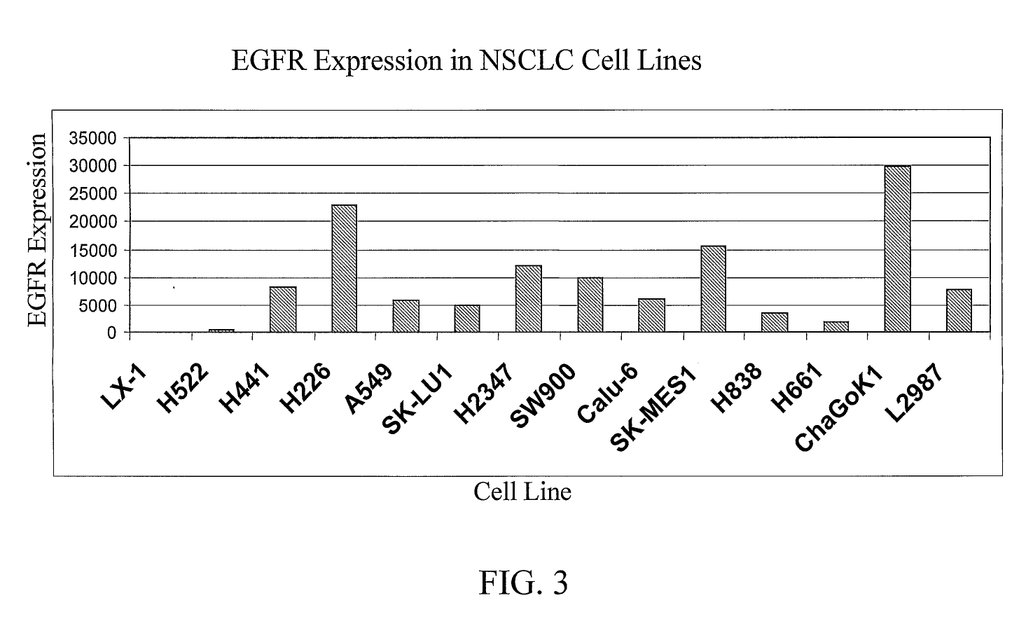

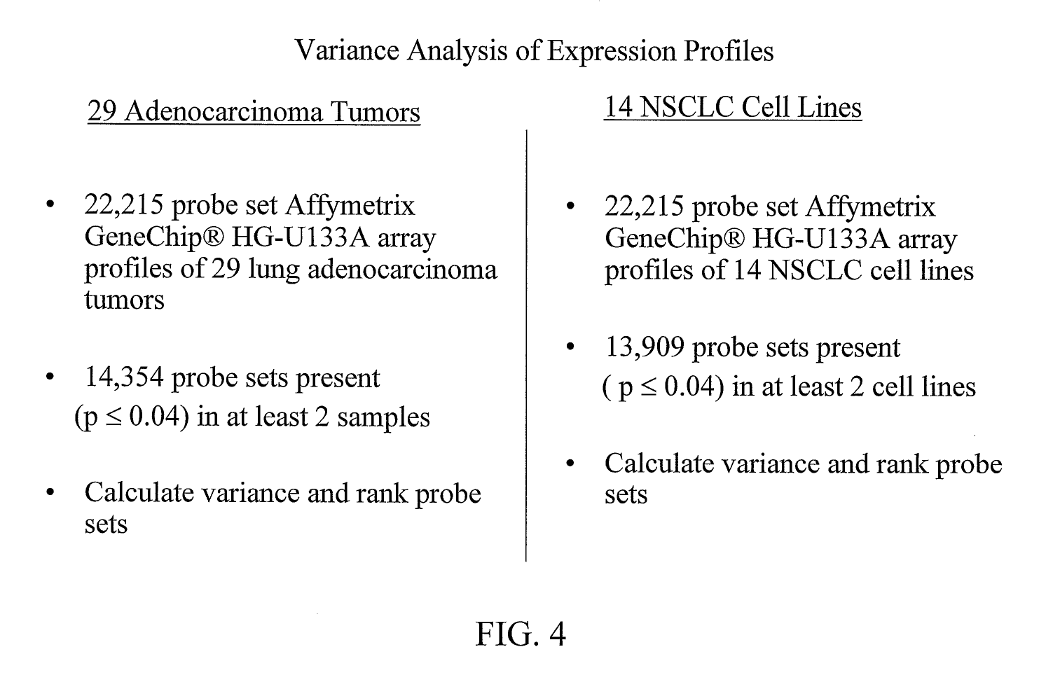

Gene sequences with a WSpread (90-10) metric<30 were removed leaving 4167 gene sequences in the adenocarcinoma tumors (FIG. 3) and 4274 gene sequences in the cell lines (FIG. 4).

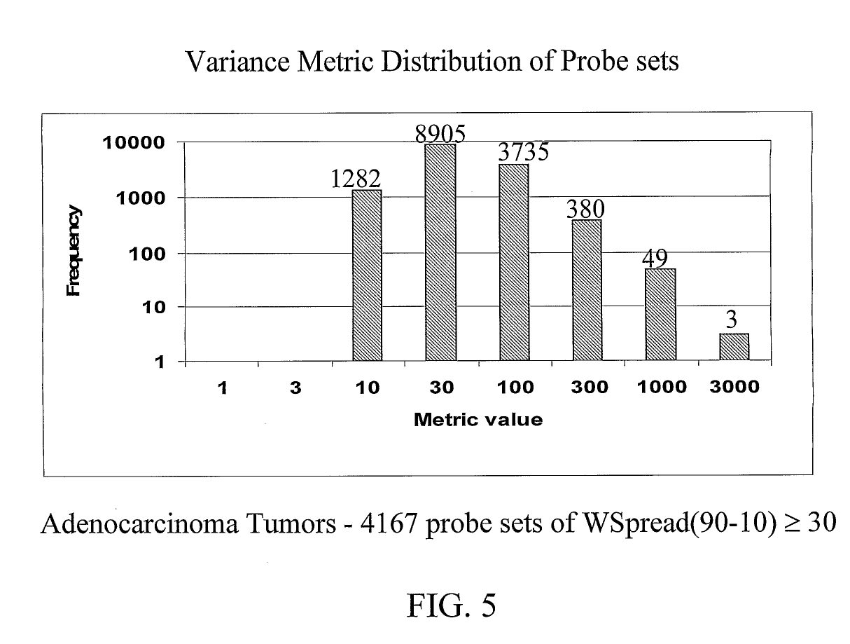

Next, the same expression filter was applied to the remaining 4167 gene sequences using the NSCLC cell line data, resulting in 3572 gene sequences for analysis. This was followed by the application of the same variance metric filter leaving 2496 gene sequences for analysis. Of the 2496 gene sequences, 776 genes sequences ranked in the top 1000 in the cell line variance analysis. These 776 sequences were chosen for further statistical analysis. The 776 gene sequences were subjected to a two-sided unequal variance t-test using the resistance/sensitivity classifications of the cell lines described above (FIG. 1). 147 gene sequences showed a significantly different expression profile between the sensitive and resistant cell lines with a p-value of <0.05 (FIG. 5). Table 1 provides a list of the 147 gene sequences identified using the two-sided unequal variance T-test. These 147 gene sequences (probe sets) represent 124 biomarkers with regard to the Unigene Titles.

A variation of the gene filtering scheme illustrated in FIG. 1 was conducted and is illustrated in FIG. 2. In this scheme, 343 gene sequences ranked in the top 1000 in both the tumor and cell line variance analysis, a total of 343 out of the 776 genes sequences, were subjected to a two-sided unequal variance T-test. 59 gene sequences showed a significantly different expression profile between the sensitive and resistant cell lines with a p-value of <0.05. These 59 biomarkers are provided in Table 1 as the first 59 biomarkers, i.e., SEQ ID NOS:1-59 and 148-206.

### Example 2

### Experimental Validation of Biomarker Candidates: Cell Line Induction Studies

Regulation by EGFR inhibitors in drug treated cell lines would lend additional support to the candidate biomarkers as being predictive of response. Induction experiments were carried out in two sensitive cell lines ChagoK1 (sensitive to cetuximab and gefitinib) and L2987 (sensitive to cetuximab, resistant to gefitinib). Induction experiments were also carried out in four cell lines that were resistant to both EGFR inhibitors: A549 and H226 (EGFR+) and LX-1 and H522 (EGFR negative) cell lines.

Cells were seeded in 6-well tissue culture dishes in DMEM supplemented with 10% FBS (Invitrogen, Carlsbad, Calif.). Twenty-four hours later the cells were switched to DMEM containing 0.5% FBS. The next day cells were treated with either 4 μg/ml cetuximab or 1 μM gefitinib. Twenty-four hours later cells were stimulated with 100 ng/ml human recombinant epidermal growth factor EGF (Biosource International, Camarillo, Calif.) for 6 hours. The cells were lysed directly in the culture dish and RNA isolation was carried out using the RNeasy mini kit (Qiagen, Valencia, Calif.). Profiling was done on U133A GeneChips (Affymetrix, Santa Clara, Calif.). Data was analyzed using GeneChip® Expression Analysis software MAS 5.0 (Affymetrix, Santa Clara, Calif.). Anova analysis of profiling data was done with PartekPro pattern recognition software (Partek, St. Charles, Miss.) using quantile normalized Affymetrix MAS5.0 values for signal intensity.

Of the 147 probe sets examined, 21 probe sets representing 18 different biomarkers (provided below in Table 3) were highly regulated (Bonferroni p<0.05 in Anova analysis) upon EGFR inhibitor treatment and/or EGF stimulation in the sensitive cell lines.

TABLE 3
Biomarkers Highly Regulated by EGFR Inhibitor Treatment
and/or EGF Stimulation in the Sensitive Cell Lines

| Unigene title and | | Affymetrix |
|---|---|---|
| SEQID NO: | Affymetrix Description | Probe Set |
| DKK1: dickkopf | gb: NM_012242.1 /DEF = Homo sapiens | 204602_at |
| homolog 1 | dickkopf (Xenopus laevis) homolog 1 (DKK1), | |
| (LOC22943) | mRNA. /FEA = mRNA /GEN = DKK1 | |
| SEQ ID NOS: 7 | /PROD = dickkopf (Xenopus laevis) homolog 1 | |
| (DNA) and 154 | /DB_XREF = gi: 7110718 /UG = Hs.40499 | |
| (amino acid) | dickkopf (Xenopus laevis) homolog 1 | |
| | /FL = gb: AF127563.1 gb: AF177394.1 | |
| | gb: NM_012242.1 | |
| S100A9: S100 | gb: NM_002965.2 /DEF = Homo sapiens S100 | 203535_at |
| calcium-binding | calcium-binding protein A9 (calgranulin B) | |
| protein A9 | (S100A9), mRNA. /FEA = mRNA | |
| (LOC6280) | /GEN = S100A9 /PROD = S100 calcium-binding | |
| SEQ ID NOS: 10 | protein A9 /DB_XREF = gi: 9845520 | |
| (DNA) and 157 | /UG = Hs.112405 S100 calcium-binding protein | |
| (amino acid) | A9 (calgranulin B) /FL = gb: M26311.1 | |
| | gb: NM_002965.2 | |
| SFN: stratifin | Cluster Incl. X57348: H. sapiens mRNA (clone | 33322_i_at |
| (LOC2810) | 9112) /cds = (165,911) /gb = X57348 /gi = 23939 | |
| SEQ ID NOS: 11 | /ug = Hs.184510 /len = 1407 | |
| (DNA) and 158 | | |
| (amino acid) | | |
| PBEF: pre-B-cell | Consensus includes gb: BF575514 /FEA = EST | 217738_at |
| colony-enhancing | /DB_XREF = gi: 11649318 | |
| factor isoform a | /DB_XREF = est: 602133090F1 | |
| (LOC10135) | /CLONE = IMAGE: 4288079 /UG = Hs.239138 | |
| SEQ ID NOS: 36 | pre-B-cell colony-enhancing factor | |
| (DNA) and 183 | /FL = gb: U02020.1 gb: NM_005746.1 | |
| (amino acid) | | |
| SERPINE2: | Consensus includes gb: AL541302 /FEA = EST | 212190_at |
| plasminogen activator | /DB_XREF = gi: 12872241 | |
| inhibitor type 1, | /DB_XREF = est: AL541302 | |
| member 2 | /CLONE = CS0DE006YI10 (5 prime) | |
| (LOC5270) | /UG = Hs.21858 trinucleotide repeat containing 3 | |
| SEQ ID NOS: 38 | | |
| (DNA) and 185 | | |
| (amino acid) | | |
| SFN: stratifin | Cluster Incl. X57348: H. sapiens mRNA (clone | 33323_r_at |
| (LOC2810) | 9112) /cds = (165,911) /gb = X57348 /gi = 23939 | |
| SEQ ID NOS: 41 | /ug = Hs.184510 /len = 1407 | |
| (DNA) and 188 | | |
| (amino acid) | | |
| IL8: interleukin 8 | gb: AF043337.1 /DEF = Homo sapiens | 211506_s_at |
| (LOC3576) | interleukin 8 C-terminal variant (IL8) mRNA, | |
| SEQ ID NOS: 44 | complete cds. /FEA = mRNA /GEN = IL8 | |
| (DNA) and 191 | /PROD = interleukin 8 C-terminal variant | |
| (amino acid) | /DB_XREF = gi: 12641914 /UG = Hs.624 | |
| | interleukin 8 /FL = gb: AF043337.1 | |
| CTSC: cathepsin C | gb: NM_001814.1 /DEF = Homo sapiens | 201487_at |
| isoform a | cathepsin C (CTSC), mRNA. /FEA = mRNA | |
| preproprotein | /GEN = CTSC /PROD = cathepsin C | |
| (LOC1075) | /DB_XREF = gi: 4503140 /UG = Hs.10029 | |
| SEQ ID NOS: 46 | cathepsin C /FL = gb: NM_001814.1 | |
| (DNA) and 193 | | |
| (amino acid) | | |
| TXNIP: thioredoxin | Consensus includes gb: AA812232 /FEA = EST | 201008_s_at |
| interacting protein | /DB_XREF = gi: 2881843 | |
| (LOC10628) | /DB_XREF = est: ob84h09.s1 | |
| SEQ ID NOS: 50 | /CLONE = IMAGE: 1338113 /UG = Hs.179526 | |
| (DNA) and 197 | upregulated by 1,25-dihydroxyvitamin D-3 | |
| (amino acid) | /FL = gb: NM_006472.1 gb: S73591.1 | |
| SAT: | gb: M55580.1 /DEF = Human | 210592_s_at |
| spermidine/spermine | spermidinespermine N1-acetyltransferase | |
| N1-acetyltransferase | mRNA, complete cds. /FEA = mRNA | |
| (LOC6303) | /GEN = spermidinespermine N1- | |
| SEQ ID NOS: 54 | acetyltransferase /PROD = spermidinespermine | |
| (DNA) and 201 | N1-acetyltransferase /DB_XREF = gi: 338335 | |
| (amino acid) | /UG = Hs.28491 spermidinespermine N1- | |
| | acetyltransferase /FL = gb: M55580.1 | |
| TXNIP: thioredoxin | gb: NM_006472.1 /DEF = Homo sapiens | 201010_s_at |
| interacting protein | upregulated by 1,25-dihydroxyvitamin D-3 | |
| (LOC10628) | (VDUP1), mRNA. /FEA = mRNA | |
| SEQ ID NOS: 57 | /GEN = VDUP1 /PROD = upregulated by 1,25- | |
| (DNA) and 204 | dihydroxyvitamin D-3 /DB_XREF = gi: 5454161 | |
| (amino acid) | /UG = Hs.179526 upregulated by 1,25- | |
| | dihydroxyvitamin D-3 /FL = gb: NM_006472.1 | |
| | gb: S73591.1 | |
| TENS1: tensin-like | gb: NM_022748.1 /DEF = Homo sapiens | 217853_at |
| SH2 domain- | hypothetical protein FLJ13732 similar to tensin | |
| containing 1 | (FLJ13732), mRNA. /FEA = mRNA | |
| (LOC64759) | /GEN = FLJ13732 /PROD = hypothetical protein | |
| SEQ ID NOS: 66 | FLJ13732 similar to tensin | |
| (DNA) and 213 | /DB_XREF = gi: 12232408 /UG = Hs.12210 | |
| (amino acid) | hypothetical protein FLJ13732 similar to tensin | |
| | /FL = gb: NM_022748.1 | |
| STK17A: | Consensus includes gb: AW194730 /FEA = EST | 202693_s_at |
| serine/threonine | /DB_XREF = gi: 6473630 | |
| kinase 17a | /DB_XREF = est: xn43d11.x1 | |
| (apoptosis-inducing) | /CLONE = IMAGE: 2696469 /UG = Hs.9075 | |
| (LOC9263) | serinethreonine kinase 17a (apoptosis-inducing) | |
| SEQ ID NOS: 69 | /FL = gb: AB011420.1 gb: NM_004760.1 | |
| (DNA) and 216 | | |
| (amino acid) | | |
| TUBB-5: tubulin | gb: BC002654.1 /DEF = Homo sapiens, Similar | 209191_at |
| beta-5 (LOC84617) | to tubulin, beta, 4, clone MGC: 4083, mRNA, | |
| SEQ ID NOS: 84 | complete cds. /FEA = mRNA /PROD = Similar to | |
| (DNA) and 231 | tubulin, beta, 4 /DB_XREF = gi: 12803638 | |
| (amino acid) | /UG = Hs.274398 Homo sapiens, Similar to | |
| | tubulin, beta, 4, clone MGC: 4083, mRNA, | |
| | complete cds /FL = gb: BC002654.1 | |
| TYMS: thymidylate | gb: NM_001071.1 /DEF = Homo sapiens | 202589_at |
| synthetase | thymidylate synthetase (TYMS), mRNA. | |
| (LOC7298) | /FEA = mRNA /GEN = TYMS | |
| SEQ ID NOS: 85 | /PROD = thymidylate synthetase | |
| (DNA) and 232 | /DB_XREF = gi: 4507750 /UG = Hs.82962 | |
| (amino acid) | thymidylate synthetase /FL = gb: BC002567.1 | |
| | gb: NM_001071.1 | |
| RAI14: retinoic acid | gb: NM_015577.1 /DEF = Homo sapiens novel | 202052_s_at |
| induced 14 | retinal pigment epithelial gene (NORPEG), | |
| (LOC26064) | mRNA. /FEA = mRNA /GEN = NORPEG | |
| SEQ ID NOS: 97 | /PROD = DKFZP564G013 protein | |
| (DNA) and 244 | /DB_XREF = gi: 13470085 /UG = Hs.15165 novel | |
| (amino acid) | retinal pigment epithelial gene | |
| | /FL = gb: NM_015577.1 gb: AF155135.1 | |
| CALD1: caldesmon 1 | Consensus includes gb: AL583520 /FEA = EST | 212077_at |
| isoform 3 (LOC800) | /DB_XREF = gi: 12952562 | |
| SEQ ID NOS: 106 | /DB_XREF = est: AL583520 | |
| (DNA) and 253 | /CLONE = CS0DC024YE13 (5 prime) | |
| (amino acid) | /UG = Hs.182183 Homo sapiens mRNA for | |
| | caldesmon, 3 UTR | |
| PALM2: paralemmin | gb: NM_007203.1 /DEF = Homo sapiens A | 202760_s_at |
| 2 (LOC114299) | kinase (PRKA) anchor protein 2 (AKAP2), | |
| SEQ ID NOS: 115 | mRNA. /FEA = mRNA /GEN = AKAP2 | |
| (DNA) and 262 | /PROD = A kinase (PRKA) anchor protein 2 | |
| (amino acid) | /DB_XREF = gi: 6005708 /UG = Hs.42322 A | |
| | kinase (PRKA) anchor protein 2 | |
| | /FL = gb: AB023137.1 gb: NM_007203.1 | |
| TPM1: tropomyosin 1 | gb: Z24727.1 /DEF = H. sapiens tropomyosin | 210986_s_at |
| (alpha) (LOC7168) | isoform mRNA, complete CDS. /FEA = mRNA | |
| SEQ ID NOS: 125 | /PROD = tropomyosin isoform | |
| (DNA) and 272 | /DB_XREF = gi: 854188 /UG = Hs.77899 | |
| (amino acid) | tropomyosin 1 (alpha) /FL = gb: Z24727.1 | |
| TPM1: tropomyosin 1 | gb: M19267.1 /DEF = Human tropomyosin | 210987_x_at |
| (alpha) (LOC7168) | mRNA, complete cds. /FEA = mRNA | |
| SEQ ID NOS: 137 | /DB_XREF = gi: 339943 /UG = Hs.77899 | |
| (DNA) and 284 | tropomyosin 1 (alpha) /FL = gb: M19267.1 | |
| (amino acid) | | |
| TUBB: tubulin, beta | gb: NM_001069.1 /DEF = Homo sapiens tubulin, | 204141_at |
| polypeptide | beta polypeptide (TUBB), mRNA. | |
| (LOC7280) | /FEA = mRNA /GEN = TUBB /PROD = tubulin, | |
| SEQ ID NOS: 147 | beta polypeptide /DB_XREF = gi: 4507728 | |
| (DNA) and 294 | /UG = Hs.179661 tubulin, beta polypeptide | |
| (amino acid) | /FL = gb: BC001194.1 gb: NM_001069.1 | |

It appears that these biomarkers are likely to be directly or indirectly involved in the EGFR signaling pathway, based on their expression modulation by EGF and/or or EGFR inhibitor treatment.

### Example 3

### Experimental Validation of Biomarker Candidates: Drug Treatment Studies in Lung Xenograft Models

Regulation by EGFR inhibitors in lung xenograft models would lend additional support to the candidate markers, as being predictive of response. Drug treatment experiments were carried out in the L2987 (sensitive to cetuximab and gefitinib), A549 (borderline sensitive to cetuximab and gefitinib), and LX1 (resistant to cetuximab and gefitinib) lung xenograft models.

In Vivo Antitumor Testing

Tumors were propagated in nude mice as subcutaneous (sc) transplants using tumor fragments obtained from donor mice. Tumor passage occurred approximately every two to four weeks. Tumors were then allowed to grow to the pre-determined size window (usually between 100-200 mg, tumors outside the range were excluded) and animals were evenly distributed to various treatment and control groups. Animals were treated with cetuximab (1 mg/mouse, q3d×10, 14; ip) or gefitinib (200 mg/kg, q1d14, 14; po). Treated animals were checked daily for treatment related toxicity/mortality. Each group of animals was weighed before the initiation of treatment (Wt1) and then again following the last treatment dose (Wt2). The difference in body weight (Wt2-Wt1) provided a measure of treatment-related toxicity. Tumor response was determined by measurement of tumors with a caliper twice a week, until the tumors reached a predetermined target size of 1 gm or became necrotic. Tumor weights (mg) were estimated from the formula:
Tumor weight=(length×width²)/2
Antitumor activity was determined in terms of primary tumor growth inhibition. This was determined in two ways: (i) calculating the relative median tumor weight (MTW) of treated (T) and control (C) mice at various time points (effects were expressed as % T/C); and (ii) calculating the tumor growth delay (T-C value), defined as the difference in time (days) required for the treated tumors (T) to reach a predetermined target size compared to those of the control group (C). Statistical evaluations of data were performed using Gehan's generalized Wilcoxon test for comparisons of time to reach tumor target size (Gehan 1965). Statistical significance was declared at p<0.05. Antitumor activity was defined as a continuous MTW % T/C≦50% for at least 1 tumor volume doubling time (TVDT) any time after the start of treatment, where TVDT (tumor volume doubling time)=median time (days) for control tumors to reach target size−median time (days) for control tumors to reach half the target size. In addition, treatment groups had to be accompanied by a statistically significant tumor growth delay (T-C value) (p<0.05) to be termed active.

Treated animals were checked daily for treatment related toxicity/mortality. When death occurred, the day of death was recorded. Treated mice dying prior to having their tumors reach target size were considered to have died from drug toxicity. No control mice died bearing tumors less than target size. Treatment groups with more than one death caused by drug toxicity were considered to have had excessively toxic treatments and their data were not included in the evaluation of the compound's antitumor efficacy.

Drug Treatment Experiments

L2987 and A549 xenograft animals were dosed with a single dose of either (1) 1 mg/mouse cetuximab, ip; (2) 250 mg/kg gefitinib, po; (3) PEG400/H₂O vehicle, po or 4) PBS vehicle, ip. Each dose was given to three independent mice. At 3 h and 24 h post-treatment the animals were sacrificed and tumors were excised and immediately placed into RNAlater solution (Qiagen, Valencia, Calif.).

RNA was isolated from the tumors using the RNeasy kits (Qiagen, Valencia, Calif.). The quality and concentration of total RNA was determined as described previously. Profiling was done on U133A GeneChips (Affymetrix, Santa Clara, Calif.). Data was analyzed using GeneChip® Expression Analysis software MAS 5.0 (Affymetrix, Santa Clara, Calif.). Anova analysis of profiling data was done with PartekPro pattern recognition software (Partek, St. Charles, Miss.) using quantile normalized Affymetrix MAS5.0 values for signal intensity.

Out of 147 probesets examined, 4 probesets representing 3 genes are significantly regulated (p<0.005 in Anova analysis) upon EGFR inhibitor treatment in the sensitive L2987 xenograft but not in the borderline sensitive A549 xenograft. The three genes are jumping translocation breakpoint (JTB), 3-phosphoadenosine 5-phosphosulfate synthase 2 (PAPSS2) and serine protease inhibitor, Kunitz type 1 (SPINT1). It appears that these biomarkers are likely to be directly or indirectly involved in the EGFR signaling pathway, based on their expression modulation by EGFR inhibitor treatment.

### Example 4

### Immunohistochemistry (IHC) Assays in Clinical Samples

Of the 147 probe sets identified preclinically, S100A9 (Calgranulin B) was chosen to examine whether there was any correlation between expression of a particular protein in the clinical samples and Best Clinical Response data.

Basic IHC Method

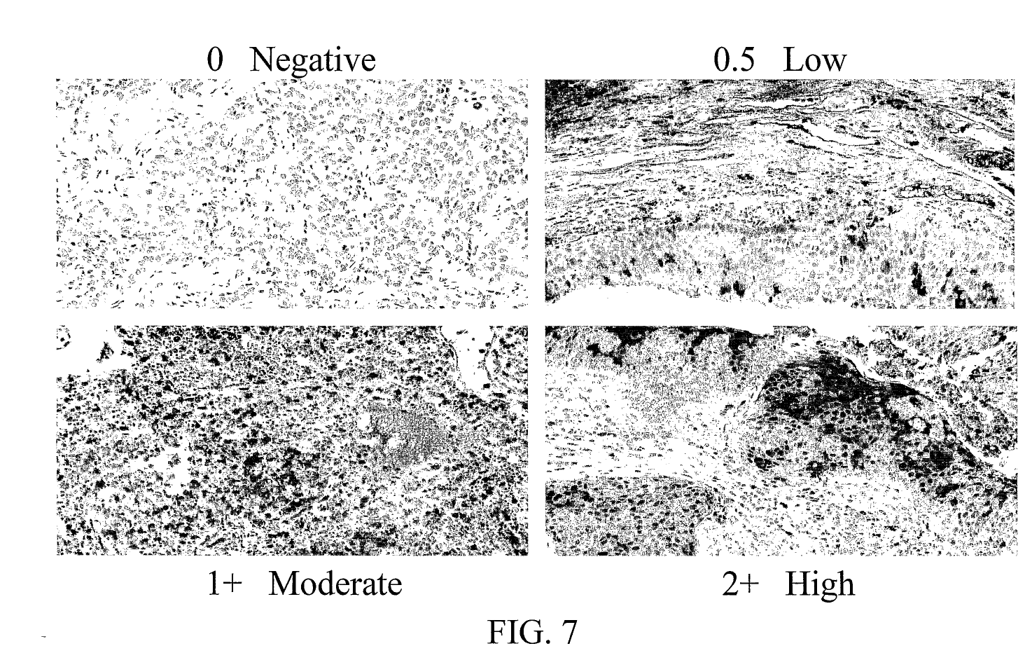

Formalin-fixed, paraffin-embedded tissues were available on slides in 5 μm sections. The sections were deparaffinized with standard xylene and hydrated through graded alcohols into water. Antigen retrieval was performed using proteinase K. Staining was done at room temperature on an automatic staining workstation TechMate 1000 (BioTek Solutions/Ventana Medical Systems, Tucson, Ariz.) by using the Envision peroxidase mouse system (DakoCytomation, Carpinteria, Calif.). Slides were placed three times for 2.5 minutes each in a hydrogen peroxide blocking medium and then allowed to react with mouse anti-human Calgranulin B monoclonal antibody (Bachem Biomedical, Germany) for 60 minutes. Immunodetection was performed with the Envision system by placing slides three times for 5 minutes each in diaminobenzidine (DAB) chromogen substrate. Counterstaining with hematoxylin for 1 minute was the final step. After staining, slides were dehydrated through an alcohol series to absolute ethanol followed by xylene rinses. Slides were permanently coverslipped with glass coverslips and permount medium. Slides were examined under a microscope to assess staining. Positive staining is indicated by the presence of a dark brown chromogen (DAB-Horse Radish Peroxidase reaction product). Hematoxylin counterstain provides a blue nuclear stain to assess cell and tissue morphology. Appropriate positive and negative controls were used. The slides were viewed randomly, without clinical data, by two independent evaluators and scored. A simple scoring system was used to reflect whether a tissue is positive or negative for the marker and to indicate the relative level of staining. A scoring scheme of negative, low, moderate or high was used to indicate the relative percentage of tumor cells staining within the tissues (FIG. 7). The scoring system simply provides an indication of relative expression of a target from tissue to tissue.

Clinical Materials and Criteria for Response

Formalin-fixed paraffin embedded lung tumor slides were obtained from patients enrolled in a phase II trial of cetuximab. In this trial, cetuximab was used as a single agent therapy for recurrent non-small-cell lung cancer patients (unpublished). The best overall response was recorded from the start of the treatment until disease progression or recurrence. Assessment of response was performed using the RECIST criteria (Response Evaluation Criteria in Solid Tumors, Tsuchida and Therasse, 2001). A partial response (PR) described at least a 30% decrease in the sum of the longest diameter (LD) of target lesions, taking as reference the baseline sum LD. Progressive disease (PD) referred to a 20% or greater increase in the sum of the LD of target lesions, taking as reference the smallest sum LD recorded since the treatment started or the appearance of new lesions. Stable Disease (SD) was used to describe neither sufficient shrinkage to qualify for PR nor sufficient increase to qualify for PD.

Calgranulin B IHC Assay on Clinical FFPET Slides

Calgranulin B IHC assay was performed on FFPET slides from 39 patients enrolled in the phase II trial of cetuximab in recurrent NSCLC patients (Table 4). Of the 39 patients, 10 were excluded from further analysis because there was no detectable tumor specimen on the slide. The remaining 29 patients that were scored for Calgranulin B staining comprised of 2 PR, 12 SD and 15 PD non-responders based on the clinical response data. The 39 samples used in this IHC analysis were derived from patients for whom tissue samples were available and from whom an informed consent could be obtained. It should be noted that the response data shown here may not reflect the response rate in the entire study.

Of the 29 patients' slides, 22 were scored as 0, 3 were scored as 0.5+, 3 were scored as 1+ and 1 slide was scored as 2+. Overall 24% of the patients tested were positive for Calgranulin B staining (Table 4).

TABLE 4
IHC Assay Results

| PROGRESSIVE | | | DISEASE | | |
|---|---|---|---|---|---|
| DISEASE | | | STABILIZATION | | |
| | Best | | | Best | |
| | Clinical | | | Clinical | |
| ID | Response | IHC | ID | Response | IHC |
| L8 | PD | negative | L10 | SD | negative |
| L11 | PD | negative | L13 | SD | positive |
| L12 | PD | positive | L40 | SD | negative |
| L14 | PD | negative | L24 | SD | negative |
| L15 | PD | negative | L27 | SD | positive |
| L18 | PD | negative | L47 | SD | positive |
| L20 | PD | negative | L28 | SD | negative |
| L41 | PD | negative | L3 | SD | negative |
| L42 | PD | negative | L4 | SD | negative |
| L44 | PD | negative | L6 | SD | positive |
| L16 | PD | negative | L34 | SD | negative |
| L5 | PD | negative | L39 | SD | positive |
| L33 | PD | negative | L1 | PR | positive |
| L37 | PD | negative | L2 | PR | negative |
| L23B | PD | negative | | | |

The results are summarized in Table 5 below.

TABLE 5
IHC Assay Results Summary

| | # responders | # non- |
|---|---|---|
| | (PR + SD) | responders |
| Calgranulin B+ | 6 | 1 |
| Calgranulin B− | 9 | 13 |

Of the 7 patients that were Calgranulin B positive, 6 had disease stabilization and 1 was a non-responder having progressive disease (Table 5). The sensitivity of the assay to identify potential responders is 40% [6/(6+9)] and the specificity is 93% [13/(13+1)].

The positive predictive value of a Calgranulin B IHC assay to identify potential responders is 86% [6/(6+1)] and the negative predictive value=59% [13/(13+9)], {Chi square p value=0.03}.

Although the data set is small, these results indicate a trend for Calgranulin B positive patients to have disease stabilization.

### Example 5

### Production of Antibodies Against the Biomarkers

Antibodies against the biomarkers can be prepared by a variety of methods. For example, cells expressing a biomarker polypeptide can be administered to an animal to induce the production of sera containing polyclonal antibodies directed to the expressed polypeptides. In one aspect, the biomarker protein is prepared and isolated or otherwise purified to render it substantially free of natural contaminants, using techniques commonly practiced in the art. Such a preparation is then introduced into an animal in order to produce polyclonal antisera of greater specific activity for the expressed and isolated polypeptide.

In one aspect, the antibodies of the invention are monoclonal antibodies (or protein binding fragments thereof). Cells expressing the biomarker polypeptide can be cultured in any suitable tissue culture medium, however, it is preferable to culture cells in Earle's modified Eagle's medium supplemented to contain 10% fetal bovine serum (inactivated at about 56° C.), and supplemented to contain about 10 g/l nonessential amino acids, about 1,00 U/ml penicillin, and about 100 μg/ml streptomycin.

The splenocytes of immunized (and boosted) mice can be extracted and fused with a suitable myeloma cell line. Any suitable myeloma cell line can be employed in accordance with the invention, however, it is preferable to employ the parent myeloma cell line (SP2/0), available from the ATCC (Manassas, Va.). After fusion, the resulting hybridoma cells are selectively maintained in HAT medium, and then cloned by limiting dilution as described by Wands et al. (1981, Gastroenterology, 80:225-232). The hybridoma cells obtained through such a selection are then assayed to identify those cell clones that secrete antibodies capable of binding to the polypeptide immunogen, or a portion thereof.

Alternatively, additional antibodies capable of binding to the biomarker polypeptide can be produced in a two-step procedure using anti-idiotypic antibodies. Such a method makes use of the fact that antibodies are themselves antigens and, therefore, it is possible to obtain an antibody that binds to a second antibody. In accordance with this method, protein specific antibodies can be used to immunize an animal, preferably a mouse. The splenocytes of such an immunized animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce the formation of further protein-specific antibodies.

### Example 6

### Immunofluorescence Assays

The following immunofluorescence protocol may be used, for example, to verify EGFR biomarker protein expression on cells or, for example, to check for the presence of one or more antibodies that bind EGFR biomarkers expressed on the surface of cells. Briefly, Lab-Tek II chamber slides are coated overnight at 4° C. with 10 micrograms/milliliter (μg/ml) of bovine collagen Type II in DPBS containing calcium and magnesium (DPBS++). The slides are then washed twice with cold DPBS++ and seeded with 8000 CHO-CCR5 or CHO pC4 transfected cells in a total volume of 125 μl and incubated at 37° C. in the presence of 95% oxygen/5% carbon dioxide.

The culture medium is gently removed by aspiration and the adherent cells are washed twice with DPBS++ at ambient temperature. The slides are blocked with DPBS++ containing 0.2% BSA (blocker) at 0-4° C. for one hour. The blocking solution is gently removed by aspiration, and 125 μl of antibody containing solution (an antibody containing solution may be, for example, a hybridoma culture supernatant which is usually used undiluted, or serum/plasma which is usually diluted, e.g., a dilution of about 1/100 dilution). The slides are incubated for 1 hour at 0-4° C. Antibody solutions are then gently removed by aspiration and the cells are washed five times with 400 μl of ice cold blocking solution. Next, 125 μl of 1 μg/ml rhodamine labeled secondary antibody (e.g., anti-human IgG) in blocker solution is added to the cells. Again, cells are incubated for 1 hour at 0-4° C.

The secondary antibody solution is then gently removed by aspiration and the cells are washed three times with 400 μl of ice cold blocking solution, and five times with cold DPBS++. The cells are then fixed with 125 μl of 3.7% formaldehyde in DPBS++ for 15 minutes at ambient temperature. Thereafter, the cells are washed five times with 400 μl of DPBS++ at ambient temperature. Finally, the cells are mounted in 50% aqueous glycerol and viewed in a fluorescence microscope using rhodamine filters.

What is claimed is:

1. A method for identifying a mammal that will respond therapeutically to a method of treating cancer comprising administering an EGFR modulator, wherein the method comprises:
(a) measuring in the mammal the level of at least one biomarker selected from the biomarkers of Table 1;
(b) exposing a biological sample from said mammal to the EGFR modulator;
(c) following the exposing of step (b), measuring in said biological sample the level of the at least one biomarker,
wherein a difference in the level of the at least one biomarker measured in step (c) compared to the level of the at least one biomarker measured in step (a) indicates that the mammal will respond therapeutically to said method of treating cancer.

2. A method for identifying a mammal that will respond therapeutically to a method of treating cancer comprising administering an EGFR modulator, wherein the method comprises:
(a) exposing a biological sample from the mammal to the EGFR modulator;
(b) following the exposing of step (a), measuring in said biological sample the level of the at least one biomarker selected from the biomarkers of Table 1,
wherein a difference in the level of the at least one biomarker measured in step (b), compared to the level of the at least one biomarker in a mammal that has not been exposed to said EGFR modulator, indicates that the mammal will respond therapeutically to said method of treating cancer.
